(19) United States
(12) Patent Application Publication
(10) Pub. No.: US 2014/0275128 A1
(43) Pub. Date: Sep. 18, 2014

(54) METHOD OF PROVIDING OCULAR NEUROPROTECTION

(71) Applicant: INOTEK PHARMACEUTICALS CORPORATION, Lexington, MA (US)

(72) Inventor: William K. MCVICAR, Sudbury, MA (US)

(73) Assignee: INOTEK PHARMACEUTICALS CORPORATION, Lexington, MA (US)

(21) Appl. No.: 14/211,567

(22) Filed: Mar. 14, 2014

Related U.S. Application Data
(60) Provisional application No. 61/798,412, filed on Mar. 15, 2013.

(51) Int. Cl. C07D 473/34; A61K 31/52; A61K 45/06

(52) U.S. Cl. CPC C07D 473/34; A61K 45/06; A61K 31/52
USPC 514/263.23

(57) ABSTRACT

Provided herein are compounds of Formula I, compositions comprising an effective amount of a compound of Formula I, and methods for preventing, reducing or treating retinal ganglion cell damage comprising administering an effective amount of a purine derivative to a subject in need thereof.

### RELATED APPLICATIONS

This application claims priority to U.S. Provisional Application No. 61/798,412, filed Mar. 15, 2013. The contents of any patents, patent applications, and references cited throughout this specification are hereby incorporated by reference in their entireties.

### TECHNICAL FIELD OF THE INVENTION

Provided herein are methods of providing ocular neuroprotection in one or more subjects in need thereof. Also provided herein are uses of certain compounds in subjects for protecting, reducing or treating in retinal ganglion cell damage.

### BACKGROUND OF THE INVENTION

The loss of retinal ganglion cells is a hallmark of certain ophthalmic diseases including ischemic optic neuropathy, (ION) and glaucoma.

The retinal ganglion cells each have an axion that extends into the brain comprising the optic nerve. Retinal ganglion cell damage is any kind of injury or damage to the retinal ganglion cells brought about by ocular compression, ocular ischemia, ocular trauma, ocular inflammation, ocular infection, normal tension glaucoma, elevated intraocular pressure, diabetes, interruption in the blood circulation to the retinal ganglion cells, ocular malignancy, ocular disease or general ocular deterioration. Optic nerve damage is also called optic nerve atrophy or optic neuropathy. Optic nerve and retinal ganglion cell damage can lead to vision distortion, vision loss and blindness.

Acute and chronic animal models of optic nerve degeneration have shown the neuroprotective potential of the alpha2 adrenergic agonist brimonidine. These models include direct injury of the optic nerve (nerve crush) and models of acute and chronic ocular hypertension (Yoles et al 1999; Donello et al 2001; WoldeMussie et al 2001; Mayor-Torroglosa et al 2005; Lambert et al 2011). Brimonidine improved RGC survival in each of these models.
1. Donello J E, Padillo E U, Webster M L, et al. alpha2-Adrenoceptor agonists inhibit vitreal glutamate and aspartate accumulation and preserve retinal function after transient ischemia. J Pharmacol Exp Ther. 2001; 296:216-23
2. Mayor-Torroglosa S, De la Villa P, Rodriguez M E, et al. Ischemia results 3 months later in altered ERG, degeneration of inner layers, and deafferented tectum: neuroprotection with brimonidine. Invest Ophthalmol V is Sci. 2005; 46:3825-35.
3. WoldeMussie E, Ruiz G, Wijono M, et al. Neuroprotection of retinal ganglion cells by brimonidine in rats with laser-induced chronic ocular hypertension. Invest Ophthalmol V is Sci. 2001; 42:2849-55.
4. Yoles E, Wheeler L A, Schwartz M. Alpha2-adrenoreceptor agonists are neuroprotective in a rat model of optic nerve degeneration. Invest Ophthalmol Vis Sci. 1999; 40:65-73.
5. Vidal-Sanz M, Lafuente M P, Mayor-Torroglosa S, et al. Brimonidine's neuroprotective effects against transient ischaemia-induced retinal ganglion cell death. Eur J. Ophthalmol. 2001; 11(Suppl 2):S36-40.
6. Lambert¹ WS, Ruiz¹ L, Crish¹ SD, Wheeler² L A, Calkins¹* D J' Brimonidine prevents axonal and somatic degeneration of retinal ganglion cell neurons; Molecular Neurodegeneration 2011, 6:4 doi:10.1186/1750-1326-6-4

In a 12-month-long clinical trial, brimonidine given topically in a 0.2% solution was shown to reduce the loss in thickness of the nerve fiber layer of the retina (RNFL), where retinal ganglion cells are found, compared to treatment with timolol. This protective effect was attributed to neuroprotection due to the similar IOP changes seen between treatment groups (Tsai J C, Chang H W. Comparison of the effects of brimonidine 0.2% and timolol 0.5% on retinal nerve fiber layer thickness in ocular hypertensive patients: a prospective, unmasked study. J Ocul Pharmacol Ther. 2005; 21:475-82). In a clinical study with a longer (mean 30.0 month) follow up in patients with low pressure glaucoma, 0.2% brimonidine was shown to slow the loss of visual field compared to the timolol control (Krupin T, Liebmann J M, Greenfield D S, et al.; Low-Pressure Glaucoma Study Group. A randomized trial of brimonidine versus timolol in preserving visual function: results from the Low-Pressure Glaucoma Treatment Study. Am J. Ophthalmol. 2011; 151:671-681). There were no differences in IOP between teatment groups. The body of preclinical studies and these supporting clinical findings make brimonidine 0.2% solution the current gold standard for preventing the optic nerve neuropathy associated with vision loss in glaucoma.

Barriers to the clinical use of 0.2% brimonidine to prevent retinal ganglion cell death and consequent vision loss in patients with optic nerve neuropathies include the required dosing frequency (three times a day) and the high rate of side effects of the therapy. The FDA labeling for 0.2% brimonidine tartrate lists oral dryness, ocular hyperemia, burning and stinging, headache, blurring, foreign body sensation, fatigue/drowsiness, conjunctival follicles, ocular allergic reactions, and ocular pruritus as occurring in 10 to 30% of patients. In the Low-Pressure Glaucoma Treatment Study referenced previously, 28.3% of study participants receiving 0.2% brimonidine discontinued the study due to adverse events, compared to 11.4% of timolol-treated subjects. This poor ocular tolerability means that compliance with chronically administered 0.2% brimonidine is a major impediment to the long-term treatment required to benefit from the neuroprotective effects of the drug.

There is, therefore, a need for other therapeutic agents that can (i) prevent, (ii) stop the progression of retinal ganglion cell/optic nerve damage and/or (iii) reverse retinal ganglion cell/optic nerve damage.

### SUMMARY OF THE INVENTION

Provided herein are selective adenosine A₁ agonist compounds, pharmaceutical compositions comprising such compounds, and methods of using such compounds to treat, reduce or prevent retinal ganglion cell damage or to provide ocular neuroprotection.

Thus in a first aspect there is provided a method of preventing retinal ganglion cell damage in a subject comprising applying an effective amount of an ophthalmic pharmaceutical composition comprising a selective adenosine A₁ agonist to an eye of the subject.

In a second aspect, the present invention provides a method of reducing retinal ganglion cell damage in a subject by administering an effective amount of an ophthalmic pharmaceutical composition comprising a selective A₁ agonist to an affected eye of the subject.

In a third aspect, the present invention provides a method of providing ocular neuroprotection in a subject in need thereof, comprising the step of: applying a pharmaceutical composition comprising an effective amount of a selective A₁ agonist to an eye of the subject.

In certain embodiments, the methods of the ophthalmic composition comprises an effective amount of a selective adenosine A₁ agonist compound according to Formula I,

[chemical structure]

or a pharmaceutically acceptable salt thereof,
wherein

A is —CH₂ONO₂, —CH₂OH, or —CH₂OSO₃H;

B and C are —OH; and

D is

[chemical structure]

In certain embodiments of the methods of the invention, the adenosine A₁ agonist is Compound A.

The methods of the invention are useful for preventing or reducing retinal ganglion cell damage or providing ocular neuroprotection in subjects having or at risk for developing diseases and conditions giving rise to the retinal ganglion cell damage including, but not limited to, ocular compression, ocular ischemia, ocular trauma (e.g., Purtsher's retinopathy), ocular inflammation, ocular infection, elevated intraocular pressure, diabetes, interruption in the blood circulation to the retinal ganglion cells, ocular malignancy, ocular disease or general ocular deterioration, glaucoma (e.g., normal tension glaucoma, pseudo-exfoliative and pigment dispersion glaucoma, and closed angle glaucoma), ocular ischemic syndrome, retinal ischemia (e.g., retinal hypoxia ischemia), retinal vein occlusion, retinal artery occlusion, diabetic retinopathy, age-related macular degeneration, visual loss from retinal detachment, conditions resulting in increased permeability of the blood-retinal barrier (BRB) resulting in fluid accumulation and retinal edema, or combinations thereof.

In one embodiment the diseases or conditions giving rise to the retinal ganglion cell damage is not caused solely by elevated intraocular pressure.

When practicing the methods of the invention, the selective adenosine A1 agonist can be administered in drops, e.g., 1 to 2 drops. In one embodiment of this method, the IOP of the affected eye is reduced by at least 10%, e.g., at least 10-20%, e.g., by 20% or more. In one embodiment the IOP of the affected eye is reduced by at least 10% for more than 3 hours, e.g., at least 10-20% for more than 3 hours, e.g., by 20% or more for more than 3 hours. In one embodiment the IOP of the affected eye is reduced by at least 10% for at least 6 hours. In one embodiment, the IOP of the affected eye is reduced by at least 20% for at least 12 hours. In one embodiment, the IOP of the affected eye is reduced by at least 20% for about 12 to about 24 hours.

In some embodiments, of the methods described herein, the effective amount of the selective adenosine A₁ agonist applied to the eye is about 20 μg to about 7.0 mg. In some embodiments, the effective amount of the selective adenosine A₁ agonist is from about 30 μg to 1 mg. In some embodiments the effective amount of selective adenosine A₁ agonist is at least 20 μg. In some embodiments, the effective amount of the selective adenosine A₁ agonist is between 60 μg and 1500 μg; is about 100 μg, about 200 μg, about 250 μg, about 300 μg, about 350 μg, about 400 μg, about 450 μg, about 500 μg, about 550 μg or about 600 μg or about 500 to 1500 μg. In certain embodiments, the effective amount of the selective adenosine A₁ agonist is about 500 μg.

In one embodiment the selective adenosine A₁ agonist is administered at an effective dose of about 0.1 to about 5.0% (w/v). In one embodiment, the selective adenosine A₁ agonist is administered at an effective dose of about 0.5 to about 1.5% (w/v). In one embodiment, the selective adenosine A₁ agonist is administered at an effective dose of about 1.0% to about 3.0% (w/v). In one embodiment, selective adenosine A₁ agonist is administered at an effective dose of about 3.0% (w/v).

In one embodiment the effective amount of the selective adenosine A1 agonist is administered as a single dose. In another embodiment, the effective amount of the selective adenosine A₁ agonist is administered as a twice daily dose. In another embodiment, the selective adenosine A1 agonist is administered 1 to 4 times daily.

In one embodiment the method may further comprise administering a second ophthalmic agent in addition to a compound of Formula I as defined above. The second ophthalmic agent can be selected from the group comprising: β-blockers, prostaglandin analogs, carbonic anhydrase inhibitors, rho-kinase inhibitors, α₂ adrenergic agonists, miotics, neuroprotectants, adenosine A₃ antagonists, adenosine A_{2A} agonists, ion channel modulators and combinations thereof.

In certain embodiments, the selective adenosine A1 agonist administered is selected from the group consisting of:
((2R,3S,4R,5R)-5-(6-(cyclopentylamino)-9H-purin-9-yl)-3,4-dihydroxytetrahydrofuran-2-yl)methyl nitrate;
((2R,3S,4R,5R)-5-(2-chloro-6-(cyclopentylamino)-9H-purin-9-yl)-3,4-dihydroxytetrahydrofuran-2-yl)methyl nitrate; sodium ((2R,3S,4R,5R)-5-(6-(cyclopentylamino)-9H-purin-9-yl)-3,4-dihydroxytetrahydrofuran-2-yl)methyl sulfate;
((2R,3S,4R,5R)-3,4-dihydroxy-5-(6-(tetrahydrofuran-3-ylamino)-9H-purin-9-yl)tetrahydrofuran-2-yl)methyl nitrate;
((2R,3S,4R,5R)-5-(6-(cyclohexylamino)-9H-purin-9-yl)-3,4-dihydroxytetrahydrofuran-2-yl)methyl nitrate;
((2R,3S,4R,5R)-5-(6-(bicycle-[2.2.1]-heptan-2-ylamino)-9H-purin-9-yl)-3,4-dihydroxytetrahydrofuran-2-yl)methyl nitrate;
sodium ((2R,3S,4R,5R)-5-(2-chloro-6-(cyclohexylamino)-9H-purin-9-yl)-3,4-dihydroxytetrahydrofuran-2-yl)methyl sulfate;
((2R,3S,4R,5R)-5-(2-chloro-6-(cyclohexylamino)-9H-purin-9-yl)-3,4-dihydroxytetrahydrofuran-2-yl)methyl nitrate;
cyclohexyladenosine (CHA) and
2-chlorocyclopentyladenosine (CCPA) and
cyclopentyladenosine (CPA).

In one embodiment the formulation comprises about 7 mg/ml of a compound of Formula I selected from:
((2R,3S,4R,5R)-5-(2-chloro-6-(cyclopentylamino)-9H-purin-9-yl)-3,4-dihydroxytetrahydrofuran-2-yl)methyl nitrate;
   and
((2R,3S,4R,5R)-3,4-dihydroxy-5-(6-(tetrahydrofuran-3-ylamino)-9H-purin-9-yl)tetrahydrofuran-2-yl)methyl nitrate.

It is to be further appreciated that the use of compounds of Formula I as defined above, or ophthalmic compositions as defined above may be used for manufacture of a medicament for preventing, reducing or treating retinal ganglion cell damage in an affected eye of a subject.

It is to be further appreciated that the use of compounds of Formula I as defined above, or ophthalmic compositions as defined above may be used for manufacture of a medicament for providing ocular neuroprotection in an affected eye of a subject.

The foregoing brief summary broadly describes the features and technical advantages of certain embodiments of the present invention. Further technical advantages will be described in the detailed description of the invention that follows. Novel features which are believed to be characteristic of the invention will be better understood from the detailed description of the invention when considered in connection with any accompanying figures and examples. However, the figures and examples provided herein are intended to help illustrate the invention or assist with developing an understanding of the invention, and are not intended to be definitions of the invention's scope.

### BRIEF DESCRIPTION OF THE DRAWINGS

FIG. 1 shows schematically a retinal cross section depicting the layers and cells that make up the retina.

FIG. 2 shows the histology data showing the degree of retinal thinning in the various ganglion cell layers along with a comparison between the effects of brimonidine and Compound A (designated as ‘Drug’ in the Figure).

FIG. 3 shows histology data for the percentage of degree of protection across the various ganglion cell layers, along with a comparison between the effects of brimonidine and Compound A (designated as ‘Drug’ in the Figure).

FIG. 4 shows the percent protection gained from the presence of Compound A or brimonidine on RGC from an ischemic insult versus an assumption of no protection in the vehicle treated group.

### DETAILED DESCRIPTION OF THE INVENTION

Embodiments of the present invention provide compounds useful for preventing, reducing or treating retinal ganglion cell damage.

Adenosine is a purine nucleoside that modulates many physiologic processes. Cellular signaling by adenosine occurs through four adenosine receptor subtypes: A₁, A_{2A}, A_{2B}, and A₃ as reported by Ralevic and Burnstock (Pharmacol Rev. 50:413-492, 1988) and Fredholm B B et al. (Pharmacol Rev. 53:527-552, 2001). In the eye, adenosine A₁ receptor agonists lower IOP in mice, rabbits and monkeys (Tian B et al. Exp Eye Res. 64:979-989, 1997; Crosson C E. J Pharmacol Exp Ther. 273: 320-326, 1995; and Avila M Y et al. Br J. Pharmacol. 134:241-245, 2001). While other publications have noted that adenosine A1 receptor agonists in the eye target the conventional outflow pathway via the trabecular meshwork (Husain S et al. J Pharmacol Exp Ther. 320: 258-265, 2007), reduction of IOP via other pathways has not been excluded.

Compounds that act as selective adenosine A1 agonists are known and have shown a variety of utilities. Selective adenosine A1 agonists have been discovered to reduce IOP in humans in clinical studies as published in PCT/US2010/033112.

In particular, described herein are compounds of Formula I (e.g., Compounds A, B, C, D, E, F, G, H, I, J or K) that can prevent, treat or reduce retinal ganglion cell damage in a subject (e.g., a human) in need thereof or provide ocular neuroprotection in a subject in need thereof.

Compounds of Formula I have the following structure:

[chemical structure]

or a pharmaceutically acceptable salt thereof,
wherein

A is —CH₂ONO₂, —CH₂OH, or —CH₂OSO₃H;

B and C are —OH;

D is

[chemical structure]

A and B are trans with respect to each other;

B and C are cis with respect to each other;

C and D are cis or trans with respect to each other;

R¹ is —H, —C₁-C₁₀ alkyl, -aryl, -3- to 7-membered monocyclic heterocycle, -8- to 12-membered bicyclic heterocycle, —C₃-C₈ monocyclic cycloalkyl, —C₃-C₈ monocyclic cycloalkenyl, —C₈-C₁₂ bicyclic cycloalkyl, —C₈-C₁₂ bicyclic cycloalkenyl-(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), or —(CH₂)ₙ-aryl;

R² is —H, halo, —CN, —NHR⁴, —NHC(O)R⁴, —NHC(O)OR⁴, —NHC(O)NHR⁴, —NHNHC(O)R⁴, —NHNHC(O)OR⁴, —NHNHC(O)NHR⁴, or —NH—N═C(R⁶)R⁷;

R⁴ is —C₁-C₁₅ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —C≡C—(C₁-C₁₀ alkyl) or —C≡C-aryl;

R⁶ is —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ—(C₃-C₈ monocyclic cycloalkenyl), -phenylene-(CH₂)ₙCOOH, or -phenylene-(CH₂)ₙCOO—(C₁-C₁₀ alkyl);

R⁷ is —H, —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkenyl) or —(CH₂)ₙ—(C₈-C₁₂ bicyclic cycloalkyl); and

each n is independently an integer ranging from 1 to 5, and a pharmaceutically acceptable vehicle.

In a further embodiment, the compounds for use in the invention are compounds having the formula

[chemical structure]

or a pharmaceutically acceptable salt thereof,
wherein

A is —CH₂ONO₂, —CH₂OH, or —CH₂OSO₃H;

B and C are —OH;

D is

[chemical structure]

A and B are trans with respect to each other;

B and C are cis with respect to each other;

C and D are cis or trans with respect to each other;

R¹ is —C₃-C₈ monocyclic cycloalkyl, -3- to 7-membered monocyclic heterocycle, or —C₈-C₁₂ bicyclic cycloalkyl; and

R² is —H or -halo.

In a further embodiment, the compounds for use in the invention are compounds having the formula

[chemical structure]

or a pharmaceutically acceptable salt thereof,
wherein

A is —CH₂ONO₂;

B and C are —OH;

D is

[chemical structure]

A and B are trans with respect to each other;

B and C are cis with respect to each other;

C and D are cis or trans with respect to each other;

R¹ is —C₃-C₈ monocyclic cycloalkyl, -3- to 7-membered monocyclic heterocycle, or —C₈-C₁₂ bicyclic cycloalkyl; and

R² is —H or -halo.

In another embodiment, the compound of Formula I is one of the following compounds:

[chemical structure]

((2R,3S,4R,5R)-5-(6-(cyclopentylamino)-9H-purin-9-yl)-3,4-dihydroxytetrahydrofuran-2-yl)methyl nitrate,

[chemical structure]

((2R,3 S,4R,5R)-5-(2-chloro-6-(cyclopentylamino)-9H-purin-9-yl)-3,4-dihydroxytetrahydrofuran-2-yl)methyl nitrate,

[chemical structure]

sodium ((2R,3S,4R,5R)-5-(6-(cyclopentylamino)-9H-purin-9-yl)-3,4-dihydroxytetrahydrofuran-2-yl)methyl sulfate,

[chemical structure]

((2R,3S,4R,5R)-3,4-dihydroxy-5-(6-(tetrahydrofuran-3-ylamino)-9H-purin-9-yl)tetrahydrofuran-2-yl)methyl nitrate,T

[chemical structure]

((2R,3S,4R,5R)-5-(6-(cyclohexylamino)-9H-purin-9-yl)-3,4-dihydroxytetrahydrofuran-2-yl)methyl nitrate,

[chemical structure]

((2R,3S,4R,5R)-5-(6-(bicycle-[2.2.1]-heptan-2-ylamino)-9H-purin-9-yl)-3,4-dihydroxytetrahydrofuran-2-yl)methyl nitrate,

[chemical structure]

sodium ((2R,3S,4R,5R)-5-(2-chloro-6-(cyclohexylamino)-9H-purin-9-yl)-3,4-dihydroxytetrahydrofuran-2-yl)methyl sulfate,

[chemical structure]

((2R,3S,4R,5R)-5-(2-chloro-6-(cyclohexylamino)-9H-purin-9-yl)-3,4-dihydroxytetrahydrofuran-2-yl)methyl nitrate, and

[chemical structure]

Cyclopentyladenosine (CPA),

[chemical structure]

2-chlorocyclopentyladenosine (CCPA),

[chemical structure]

Cyclohexyladenosine (CHA),
   or pharmaceutically acceptable salts thereof.

Where discrepancies exist between a compound's name and a compound's structure, the chemical structure will control.

There are a number of methods which can be used to measure the function of retinal ganglion cells. For example, damage to retinal ganglion cells can be measured using the following techniques:

(i) measurement of visual field loss. Visual field loss and its progression are hallmarks in glaucoma, including normal tension glaucoma, and high IOP glaucoma, optic neuritis and retinal ganglion cell damage. Visual field loss can be measured using various perimetery techniques. Visual field loss measurements can be very useful in finding early changes in vision caused by RGC damage.

(ii) electroretinogram (ERG) or electroretinography measurements provide information on damage to RGC. Electroretinography measures electrical activity generated by the photoreceptor cells in the retina when the eye is stimulated by certain light sources. The measurement is captured by electrodes placed on the front surface of the eye (e.g. cornea) and the skin near the eye and a graphic record called an electroretinogram (ERG) is produced. Electroretinography is useful in diagnosing several hereditary and acquired disorders of the retina, damage to retinal ganglion cells by conditions such as but not limited retinitis pigmentosa, a detached retina or functional changes caused by arteriosclerosis or diabetes. In particular, the photopic negative response (PhNR) of an ERG is thought to measure the presence of intact, functioning RGCs (Viswanathan S, Frishman L J, Robson J G, et al. The photopic negative response of the macaque electroretinogram: reduction by experimental glaucoma. Invest Ophthalmol V is Sci. 1999; 40:1124-1136), and this signal has been shown to correlate to visual field loss in patients with glaucomatous vision field loss (Viswanathan S, Frishman U, Robson J G, Walter J W. The photopic negative response of the flash electroretinogram in primary open angle glaucoma. Invest Ophthalmol V is Sci. 2001; 42:514-522),

(iii) retinal nerve fiber layer thickness (RNFL) measurements, measured by optical coherence tomography or scanning laser polarimetry as reported in Tsai J C, Chang H W. Comparison of the effects of brimonidine 0.2% and timolol 0.5% on retinal nerve fiber layer thickness in ocular hypertensive patients: a prospective, unmasked study. J OculPharmacolTher. 2005; 21:475-82.

Subjects that are susceptible to or at risk of developing RGC damage or requiring ocular neuroprotection would be candidates for employing the preventative methods of the invention are subjects having a family history of glaucoma (e.g., normal tension glaucoma, pseudo-exfoliative and pigment dispersion glaucoma, and closed angle glaucoma); subjects that have a family history of visual field loss; subjects that have a family history of ocular ischemic syndrome, retinal ischemia (e.g., retinal hypoxia ischemia), retinal vein occlusion, retinal artery occlusion, diabetic retinopathy, age-related macular degeneration, visual loss from retinal detachment, conditions resulting in increased permeability of the blood-retinal barrier (BRB) resulting in fluid accumulation and retinal edema; subjects that are to face ocular surgery or have experienced ocular trauma; as well as subjects that have ocular diseases or diseases associated with the development of retinal ganglion cell damage including glaucoma (e.g., normal tension glaucoma, pseudo-exfoliative and pigment dispersion glaucoma, and closed angle glaucoma), diabetes, malignancy, infection, ocular ischemia, ocular inflammation, ocular compression, elevated intraocular pressure, interruption in the blood circulation to the retinal ganglion cells, ocular ischemic syndrome, retinal ischemia (e.g., retinal hypoxia ischemia), retinal vein occlusion, retinal artery occlusion, diabetic retinopathy, age-related macular degeneration, visual loss from retinal detachment, conditions resulting in increased permeability of the blood-retinal barrier (BRB) resulting in fluid accumulation and retinal edema, or combinations thereof.

In one embodiment, provided herein is a method of preventing retinal ganglion cell damage, comprising administering an effective amount of a compound of Formula I to an eye of a subject.

In another embodiment, provided herein is a method of reducing or treating retinal ganglion cell damage, comprising applying an effective amount of a compound of Formula I to an affected eye of a subject.

In another embodiment, provided herein is a method of preventing, reducing or treating retinal ganglion cell damage, comprising applying an effective amount of a compound of Formula I to an eye of a subject. In another embodiment, about 0.1 to 3.0% (w/v) of a compound of Formula I is applied to an eye of a subject from 1 to 4 times daily. In one embodiment, about 0.5 to about 1.5% (w/v) of a compound of Formula I is applied to an eye of a human from 1 to 4 times daily. In still another embodiment, about 1.5% (w/v) of a compound of Formula I is applied to an eye of a human from 1 to 4 times daily. In one embodiment, the compound of Formula I is applied twice daily. In one embodiment, the compound of Formula I is applied once daily. A compound of Formula I can be administered in drops, e.g., 1 to 2 drops.

In another embodiment, provided herein is a method of preventing, reducing or treating retinal ganglion cell damage, comprising administering an effective amount of Compound A to a subject. In still another embodiment, provided herein is a method of preventing, reducing or treating retinal ganglion cell damage, comprising applying an effective amount of Compound A to an eye of a subject. In one embodiment, about 0.5 to about 1.5% (w/v) of Compound A is applied to an eye of a subject from 1 to 4 times daily. In another embodiment, about 0.5 to about 1.5% (w/v) of Compound A is applied to an eye of a subject from 1 to 4 times daily. In another embodiment, about 1.5% (w/v) of Compound A is applied to an eye of a subject from 1 to 4 times daily. In one embodiment, the compound of Formula I is applied twice daily. In one embodiment, the compound of Formula I is applied once daily. The Compound A can be administered in drops, e.g., 1 to 2 drops.

In another embodiment, provided herein is the use of a compound of Formula I for the manufacture of a medicament for preventing or treating retinal ganglion cell damage in a subject. In another embodiment, provided herein is the use of a compound of Formula I for the manufacture of a medicament for reducing retinal ganglion cell damage in a subject. In another embodiment, provided herein is the use of a compound of Formula I for the manufacture of a medicament for treating retinal ganglion cell damage in a subject.

In another embodiment, provided herein is the use of a compound of Formula I for the manufacture of a medicament for providing ocular neuroprotection in a subject.

In another embodiment, provided herein is the use of a compound of Formula I for preventing retinal ganglion cell damage in a subject. In another embodiment, provided herein is the use of a compound of Formula I for reducing retinal ganglion cell damage associated with glaucoma in a subject. In another embodiment, provided herein is the use of a compound of Formula I for providing ocular neuroprotection in a subject.

In another embodiment, provided herein is the use of a compound of Formula I for treating retinal ganglion cell damage in a subject.

In another embodiment, provided herein is the use of Compound A for preventing retinal ganglion cell damage in a subject. In another embodiment, provided herein is the use of Compound A for reducing retinal ganglion cell damage in a subject. In another embodiment, provided herein is the use of Compound A for treating retinal ganglion cell damage in a subject. In another embodiment, provided herein is the use of Compound A for providing ocular neuroprotection in a subject.

It is recognized that compounds of Formula I can contain one or more chiral centers. This invention contemplates all enantiomers, diastereomers, and mixtures of Formulas I thereof.

Furthermore, certain embodiments of the present invention comprise pharmaceutically acceptable salts of compounds according to Formula I.

Pharmaceutically acceptable salts comprise, but are not limited to, soluble or dispersible forms of compounds according to Formula I that are suitable for treatment of disease without undue undesirable effects such as allergic reactions or toxicity.

Representative pharmaceutically acceptable salts include, but are not limited to, acid addition salts such as acetate, citrate, benzoate, lactate, or phosphate and basic addition salts such as lithium, sodium, potassium, or aluminum.

### DEFINITIONS

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

As used herein, the term “selective adenosine A₁ agonist” means an A₁ agonist that has a high affinity to the A₁ receptor while simultaneously having a low affinity for the A_{2A}, and A₃ adenosine receptors. Compounds of Formula I (e.g., Compounds A to K) above have affinities to the A₁ receptor considerably greater than their respective affinities to the A_{2A} and A₃ receptors. The A₁ selectivity data for compounds A to K is summarized in the Table below.

| | | | A₁ > A₃ |
|---|---|---|---|
| | | A₁ > A_{2A} | SELECTIVITY |
| | A₁ (Ki (nm)) | SELECTIVITY | [KiA₃(nm)/ |
| Compound | POTENCY | [KiA₂(nm)/KiA₁(nm)] | KiA₁(nm)] |
| Compound A | 0.97 | 4837 | 725 |
| Compound B | 2.63 | 1593 | 195 |
| Compound C | 4.05 | 2250 | 251 |
| Compound D | 10.6 | >9434 | 202 |
| Compound E | 1.32 | 878 | 1098 |
| Compound F | 1.47 | 3945 | 260 |
| Compound G | 1.36 | 200 | 130 |
| Compound H | 8 | 192 | 167 |
| Compound I | 2.3 | 345 | 31.3 |
| (CPA) | | | |
| Compound J | 0.83 | 2735 | 50 |
| (CCPA) | | | |
| Compound K | 0.732 | 839 | 206 |
| (CHA) | | | |

As used herein, the term “alkyl” refers to a fully saturated branched or unbranched hydrocarbon moiety. Preferably the alkyl comprises 1 to 20 carbon atoms, more preferably 1 to 16 carbon atoms, 1 to 10 carbon atoms, 1 to 7 carbon atoms, or 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and the like. Furthermore, the expression “Cₓ-C_{y}-alkyl”, wherein x is 1-5 and y is 2-15 indicates a particular alkyl group (straight- or branched-chain) of a particular range of carbons. For example, the expression C₁-C₄-alkyl includes, but is not limited to, methyl, ethyl, propyl, butyl, isopropyl, tert-butyl and isobutyl. The term alkyl includes, but is not limited to, C₁-C₁₅ alkyl, C₁-C₁₀ alkyl and C₁-C₆ alkyl.

The term “C₁-C₁₅ alkyl” as used herein refers to a straight or branched chain, saturated hydrocarbon having from 1 to 15 carbon atoms. Representative C₁-C₁₅ alkyl groups include, but are not limited to methyl, ethyl, propyl, isopropyl, butyl, sec-butyl, tert-buty, pentyl, isopentyl, neopentyl, hexyl, isohexyl, neohexyl, heptyl, isoheptyl, neoheptyl, octyl, isooctyl, neooctyl, nonyl, isononyl, neononyl, decyl, isodecyl, neodecyl, undecyl, dodecyl, tridecyl, tetradecyl and pentadecyl. In one embodiment, the C₁-C₁₅ alkyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₁-C₁₅ alkyl is unsubstituted.

The term “C₁-C₁₀ alkyl” as used herein refers to a straight or branched chain, saturated hydrocarbon having from 1 to 10 carbon atoms. Representative C₁-C₁₀ alkyl groups include, but are not limited to methyl, ethyl, propyl, isopropyl, butyl, sec-butyl, tert-butyl, pentyl, isopentyl, neopentyl, hexyl, isohexyl, neohexyl, heptyl, isoheptyl, neoheptyl, octyl, isooctyl, neooctyl, nonyl, isononyl, neononyl, decyl, isodecyl and neodecyl. In one embodiment, the C₁-C₁₀ alkyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₁-C₁₀ alkyl is unsubstituted. C₁-C₁₀ alkyl includes, but is not limited to, C₁-C₆ alkyl.

The term “C₁-C₆ alkyl” as used herein refers to a straight or branched chain; saturated hydrocarbon having from 1 to 6 carbon atoms. Representative C₁-C₆ alkyl groups include, but are not limited to methyl, ethyl, propyl, isopropyl, butyl, sec-butyl, tert-buty, pentyl, isopentyl, neopentyl, hexyl, isohexyl, and neohexyl. Unless indicated, the C1-C6 alkyl is unsubstituted.

The term “aryl” as used herein refers to a phenyl group or a naphthyl group. In one embodiment, the aryl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the aryl is unsubstituted.

The term “C₃-C₈ monocyclic cycloalkyl” as used herein is a 3-, 4-, 5-, 6-, 7- or 8-membered saturated non-aromatic monocyclic cycloalkyl ring. Representative C₃-C₈ monocyclic cycloalkyl groups include, but are not limited to, cyclopropyl, cyclobutyl, cyclopentyl, cyclohexyl, cycloheptyl and cyclooctyl. In one embodiment, the C₃-C₈ monocyclic cycloalkyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₃-C₈ monocyclic cycloalkyl is unsubstituted.

The term “C₃-C₈ monocyclic cycloalkenyl” as used herein is a 3-, 4-, 5-, 6-, 7- or 8-membered non-aromatic monocyclic carbocyclic ring having at least one endocyclic double bond, but which is not aromatic. It is to be understood that when any two groups, together with the carbon atom to which they are attached form a C₃-C₈ monocyclic cycloalkenyl group, the carbon atom to which the two groups are attached remains tetravalent. Representative C₃-C₈ monocyclic cycloalkenyl groups include, but are not limited to, cyclopropenyl, cyclobutenyl, 1,3-cyclobutadienyl, cyclopentenyl, 1,3-cyclopentadienyl, cyclohexenyl, 1,3-cyclohexadienyl, cycloheptenyl, 1,3-cycloheptadienyl, 1,4-cycloheptadienyl, -1,3,5-cycloheptatrienyl, cyclooctenyl, 1,3-cyclooctadienyl, 1,4-cyclooctadienyl, -1,3,5-cyclooctatrienyl. In one embodiment, the C₃-C₈ monocyclic cycloalkenyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₃-C₈ monocyclic cycloalkenyl is unsubstituted.

The term “C₈-C₁₂ bicyclic cycloalkyl” as used herein is a 8-, 9-, 10-, 11- or 12-membered saturated, non-aromatic bicyclic cycloalkyl ring system. Representative C₈-C₁₂ bicyclic cycloalkyl groups include, but are not limited to, decahydronaphthalene, octahydroindene, decahydrobenzocycloheptene, and dodecahydroheptalene. In one embodiment, the C₈-C₁₂ bicyclic cycloalkyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₈-C₁₂ bicyclic cycloalkyl is unsubstituted.

The term “C₈-C₁₂ bicyclic cycloalkenyl” as used herein is a 8-, 9-, 10-, 11- or 12-membered non-aromatic bicyclic cycloalkyl ring system, having at least one endocyclic double bond. It is to be understood that when any two groups, together with the carbon atom to which they are attached form a C₈-C₁₂ bicyclic cycloalkenyl group, the carbon atom to which the two groups are attached remains tetravalent. Representative C₈-C₁₂ bicyclic cycloalkenyl groups include, but are not limited to, octahydronaphthalene, hexahydronaphthalene, hexahydroindene, tetrahydroindene, octahydrobenzocycloheptene, hexahydrobenzocycloheptene, tetrahydrobenzocyclopheptene, decahydroheptalene, octahydroheptalene, hexahydroheptalene, and tetrahydroheptalene. In one embodiment, the C₈-C₁₂ bicyclic cycloalkyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₈-C₁₂ bicyclic cycloalkenyl is unsubstituted.

The term “halo” as used herein refers to —F, —Cl, —Br or —I.

The term “3- to 7-membered monocyclic heterocycle” refers to: (i) a 3- or 4-membered non-aromatic monocyclic cycloalkyl in which 1 of the ring carbon atoms has been replaced with an N, O or S atom; or (ii) a 5-, 6-, or 7-membered aromatic or non-aromatic monocyclic cycloalkyl in which 1-4 of the ring carbon atoms have been independently replaced with a N, O or S atom. The non-aromatic 3- to 7-membered monocyclic heterocycles can be attached via a ring nitrogen, sulfur, or carbon atom. The aromatic 3- to 7-membered monocyclic heterocycles are attached via a ring carbon atom. Representative examples of a 3- to 7-membered monocyclic heterocycle group include, but are not limited to furanyl, furazanyl, imidazolidinyl, imidazolinyl, imidazolyl, isothiazolyl, isoxazolyl, morpholinyl, oxadiazolyl, oxazolidinyl, oxazolyl, oxazolidinyl, pyrimidinyl, phenanthridinyl, phenanthrolinyl, piperazinyl, piperidinyl, pyranyl, pyrazinyl, pyrazolidinyl, pyrazolinyl, pyrazolyl, pyridazinyl, pyridooxazole, pyridoimidazole, pyridothiazole, pyridinyl, pyrimidinyl, pyrrolidinyl, pyrrolinyl, quinuclidinyl, tetrahydrofuranyl, thiadiazinyl, thiadiazolyl, thienyl, thienothiazolyl, thienooxazolyl, thienoimidazolyl, thiomorpholinyl, thiophenyl, triazinyl, triazolyl. In one embodiment, the 3- to 7-membered monocyclic heterocycle group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the 3- to 7-membered monocyclic heterocycle is unsubstituted.

The term “8- to 12-membered bicyclic heterocycle” refers to a bicyclic 8- to 12-membered aromatic or non-aromatic bicyclic cycloalkyl in which one or both of the of the rings of the bicyclic ring system have 1-4 of its ring carbon atoms independently replaced with a N, O or S atom. Included in this class are 3- to 7-membered monocyclic heterocycles that are fused to a benzene ring. A non-aromatic ring of an 8- to 12-membered monocyclic heterocycle is attached via a ring nitrogen, sulfur, or carbon atom. An aromatic 8- to 12-membered monocyclic heterocycles are attached via a ring carbon atom. Examples of 8- to 12-membered bicyclic heterocycles include, but are not limited to, benzimidazolyl, benzofuranyl, benzothiofuranyl, benzothiophenyl, benzoxazolyl, benzthiazolyl, benztriazolyl, benztetrzolyl, benzisoxazolyl, benzisothiazolyl, benzimidazolinyl, cinnolinyl, decahydroquinolinyl, 1H-indazolyl, indolenyl, indolinyl, indolizinyl, indolyl, isobenzofuranyl, isoindazolyl, isoindolyl, isoindolinyl, isoquinolinyl, naphthyridinyl, octahydroisoquinolinyl, phthalazinyl, pteridinyl, purinyl, quinoxalinyl, tetrahydroisoquinolinyl, tetrahydroquinolinyl, and xanthenyl. In one embodiment, each ring of a the -8- to 12-membered bicyclic heterocycle group can substituted with one or more of the following groups: -halo, —O—(C₁-C₆ alkyl), —OH, —CN, —COOR′, —OC(O)R′, —N(R′)₂, —NHC(O)R′. or —C(O)NHR′ groups wherein each R′ is independently —H or unsubstituted —C₁-C₆ alkyl. Unless indicated, the 8- to 12-membered bicyclic heterocycle is unsubstituted. Representative examples of a “phenylene group” are depicted below:

The phrase “pharmaceutically acceptable salt,” as used herein, is a salt of an acid and a basic nitrogen atom of a purine compound. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. The pharmaceutically acceptable salt can also be a camphorsulfonate salt. The term “pharmaceutically acceptable salt” also refers to a salt of a purine compound having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like. The term “pharmaceutically acceptable salt” also includes a hydrate of a purine compound.

Some chemical structures herein are depicted using bold and dashed lines to represent chemical bonds. These bold and dashed lines depict absolute stereochemistry. A bold line indicates that a substituent is above the plane of the carbon atom to which it is attached and a dashed line indicates that a substituent is below the plane of the carbon atom to which it is attached.

The term “effective amount” as used herein refers to an amount of a selective adenosine A1 agonist that is effective for: (i) preventing retinal ganglion cell damage (ii) reducing retinal ganglion cell damage or (iii) treating retinal ganglion cell damage in a subject.

The term “subject” is intended to include organisms which are at risk of developing or are afflicted with a disease, disorder or condition associated with retinal ganglion cell damage. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of developing or potentially capable of suffering from retinal ganglion cell damage.

The term “treat,” “treated,” “treating” or “treatment” includes the diminishment or alleviation of at least one symptom associated or caused by the state, disorder or disease being treated. For example, the term “treat” may mean to reduce or prevent further damage or loss of retinal ganglion cells. For example, treatment can be diminishment of one or several symptoms of a disorder or complete eradication of a disorder.

The terms “protect” or “prevent” are used interchangeably herein to delay the onset (i.e., the period prior to clinical manifestation of a disease) and/or to reduce the likelihood of a subject developing or worsening of a disease (e.g., a subject at risk of developing a disease). For example, the formulations of the invention may be used to prevent elevated intraocular pressure, and/or may be used as a neuroprotective composition to prevent retinal ganglion cell damage and/or retinal ganglion cell loss.

The term “use” includes any one or more of the following embodiments of the invention, respectively: the use in the treatment of retinal ganglion cell damage; the use for the manufacture of pharmaceutical compositions for use in the treatment of the diseases or conditions giving rise to retinal ganglion cell damage, e.g., in the manufacture of a medicament; methods of use of compounds of the invention in the treatment of such diseases or conditions; pharmaceutical preparations having compounds of the invention for the treatment of diseases or conditions causing retinal ganglion cell damage; and compounds of the invention for use in the treatment of conditions and diseases that cause retinal ganglion cell damage; as appropriate and expedient, if not stated otherwise.

In particular, diseases or conditions to be treated and are thus preferred for use of a compound of the present invention include but are not limited to brought about by ocular compression, ocular ischemia, ocular trauma, ocular inflammation, ocular infection, glaucoma, elevated intraocular pressure, interruption in the blood circulation to the retinal ganglion cells, ocular malignancy, ocular disease or general ocular deterioration or combinations thereof.

The term “about” or “approximately” usually means within 20%, more preferably within 10%, and most preferably still within 5% of a given value or range. Alternatively, especially in biological systems, the term “about” means within about a log (i.e., an order of magnitude) preferably within a factor of two of a given value.

As used herein, the term “drop” refers to a quantity of ophthalmically acceptable fluid that resembles a liquid drop. In one embodiment, a drop refers to a liquid volume equivalent to about 5 μl to about 200 μl, e.g., about 30 μl to about 80 μl.

The following abbreviations are used herein and have the indicated definitions: CCPA is 2-chloro-N6-cyclopentyladenosine; CPA is N6-cyclopentyladenosine; NECA is adenosine-5′-(N-ethyl)carboxamido; NMR is nuclear magnetic resonance; R-PIA is N6-(2-phenyl-isopropyl) adenosine, R-isomer; HPβCD is hydroxypropyl β-cyclodextrin. GCL is ganglion cell layer, IPL is inner plexiform layer, INL is inner nuclear layer, OPL is outer plexiform layer, ONL is outer nuclear layer and TR is total retinal thickness.

### Methods of Synthesis

Compounds according to Formula I can be prepared by using synthetic procedures described in U.S. Pat. No. 7,423,144, the disclosure of which is incorporated herein in its entirety, as well as other published methods (see Cristalli et al., J. Med. Chem. 35:2363-2369, 1992; Cristalli et al., J. Med. Chem. 37:1720-1726, 1994; Cristalli et al, J. Med. Chem. 38:1462-1472, 1995; and Camaioni et al., Bioorg. Med. Chem. 5:2267-2275, 1997), or by using the synthetic procedures outlined below.

Scheme 1 shows methods for making nucleoside intermediates that are useful for making the compounds of the invention.

[chemical structure]

[chemical structure]

wherein R₂ is as defined above.

The protected ribose compound of Formula 1 can be coupled with a purine compound of Formula 2 using lithium hexamethyldisilazide and trimethylsilyl triflate, followed by acetonide removal using trifluoroacetic acid to provide nucleoside intermediates of Formula 3 and their corresponding other anomers of Formula 4. Similarly, the ribose diacetate of Formula 5 can be coupled with a compound of Formula 2 using lithium hexamethyldisilazide and trimethylsilyl triflate to provide acetonide-protected nucleoside intermediates of Formula 6 and their corresponding other anomers of Formula 7.

Scheme 2 shows a method useful for making the adenosine intermediates of Formula 8 which are useful for making the compounds of the invention.

[chemical structure]

where R¹ and R² are defined above.

The 6-chloroadenosine derivative of formula 3a is converted to its 2′,3′-acetonide using acetone and 2,2-dimethoxypropane in the presence of camphorsulfonic acid. The acetonide can be further derviatized using an amine of formula R¹—NH₂ in the presence of base to provide compounds of formula 8.

Methodology useful for making other compounds of the invention is described in Scheme 4.

[chemical structure]

where R¹ and R² are defined above.

The adenosine intermediates of formula 8 can be converted to their 5′-nitrate analogs using nitric acid in the presence of acetic anhydride, or other nitrating agents, such as MsCl/ONO₃ or nitrosonium tetrafluoroborate. Acetonide removal using TFA/water provides compounds of the invention.

Methodology useful for making the Purine Derivatives of Formula (Id) wherein R³ is —CH₂OSO₃H is outlined in Scheme 6.

[chemical structure]

where R¹ and R² are defined above.

The adenosine intermediates of formula 8 can be treated with sulfur trioxide-pyridine complex to provide the corresponding 5′-sulfonic acid pyridine salt intermediate. The pyridine salt intermediate can then be neutralized using NaOH or KOH, followed by acetonide removal using TFA/water to provide the corresponding sodium or potassium salt, respectively, of the Purine Derivatives of Formula (Id) wherein A is —CH₂OSO₃H. Treatment of the sodium or potassium salt with strong aqueous acid, such as sulfuric or hydrochloric acid, provides compounds of the invention wherein A is —CH₂OSO₃H.

### Modes of Delivery

The compounds according to Formula I can be incorporated into various types of ophthalmic compositions or formulations for delivery. Formula I compounds may be delivered directly to the eye (for example: topical ocular drops or ointments; slow release devices such as pharmaceutical drug delivery sponges implanted in the cul-de-sac or implanted adjacent to the sclera or within the eye; periocular, conjunctival, sub-tenons, intracameral, intravitreal, or intracanalicular injections) or systemically (for example: orally, intravenous, subcutaneous or intramuscular injections; parenterally, dermal or nasal delivery) using techniques well known by those of ordinary skill in the art. It is further contemplated that the agents of the invention may be formulated in intraocular insert or implant devices.

The compounds of Formula I are preferably incorporated into topical ophthalmic formulations with a pH of about 4-8 for delivery to the eye. Various formulations of Compound A, in particular are described in PCT/US2010/033112, PCT/US2010/054040, and co-pending U.S. Provisional Application No. 61/793,273 entitled “Ophthalmic Formulations,” filed on Mar. 15, 2013, the contents of which are herein incorporated as if individually set forth.

The compounds may be combined with ophthalmologically acceptable preservatives, surfactants, viscosity enhancers, penetration enhancers, particle stabilizers, buffers, sodium chloride, and water to form an aqueous, sterile ophthalmic suspension or solution. Ophthalmic solution formulations may be prepared by dissolving a compound in a physiologically acceptable isotonic aqueous buffer. Further, the ophthalmic solution may include an ophthalmologically acceptable surfactant to assist in dissolving the compound. Furthermore, the ophthalmic solution may contain an agent to increase viscosity or solubility such as hydroxypropyl β-Cyclodextrin (HPβCD), hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient may be combined with a preservative in an appropriate vehicle such as mineral oil, liquid lanolin, or white petrolatum. Sterile ophthalmic gel formulations may be prepared by suspending the compound in a hydrophilic base prepared from the combination of, for example, carbopol-974, or the like, according to the published formulations for analogous ophthalmic preparations; preservatives and tonicity agents can be incorporated.

Compounds in preferred embodiments are contained in a composition in amounts sufficient to prevent, reduce or treat retinal ganglion cell damage in patients experiencing elevated IOP and/or maintaining normal IOP levels in POAG or OHT patients. Such amounts are referred to herein as “an amount effective to prevent, reduce or treat retinal ganglion cell damage,” or more simply “an effective amount.” The compounds will normally be contained in these formulations in an amount of between about 0.1% and 3.0% (w/v), or between about 0.5 to about 1.5% (w/v). Thus, for topical presentation 1 to 2 drops of these formulations would be delivered to the surface of the eye from 1 to 4 times per day, according to the discretion of a skilled clinician.

The compounds of Formula I can also be used in combination with other oculartreatment agents, such as, but not limited to, β-blockers, prostaglandin analogs, carbonic anhydrase inhibitors, α₂ adrenergic agonists, miotics, and neuroprotectants adenosine A₃ antagonists, adenosine A_{2A} agonists and combinations thereof.

The invention will be further illustrated by way of the following Examples.

### Example 1

### Evaluation of Neuroprotective Effects of Compound A and Brimonidine on Ischemia-Induced Retinal Ganglion Cell Death in Long-Evans Rats

Treatment groups of 10 male Long Evans rats, six to eight weeks of age, were anesthetized by i.p. injection of a combination of ketamine (80 mg/kg) and xylazine (8 mg/kg). Additional ketamine was administered as needed to maintain appropriate anesthetic depth, and body temperature was maintained by use of an isothermal pad placed under the animal. Once under anesthesia, topical anesthetic (0.5% proparacaine hydrochloride, single drop) was applied to the cornea of the right eye. Five minutes after application of topical anesthetic, a single drop of treatment (0.2% brimonidine tartrate, Compound A 2.5% ophthalmic suspension, or Compound A vehicle) was applied to the cornea, with a second drop administered 5 minutes after the first. Several minutes after the second treatment, the anterior chamber of the right eye was cannulated with a 30G needle connected to an elevated reservoir of sterile Hanks balanced salt solution with a fluid level 59 inches above eye level (equivalent to a hydrostatic pressure of 110 mmHg) behind a closed stopcock. Five minutes after the second treatment (fifteen minutes after application of topical anesthetic), the stopcock was opened, and the rise in pressure was directly visualized by inflation of the globe. Animals that suffered an injury to the iris or cornea (beyond the single entry point) were excluded from the study. Sixty minutes after inducing the rise in pressure, the ischemic injury was terminated by closing the stopcock and removing the cannula. The needle entry wound was sealed with veterinary tissue adhesive and the animals allowed to recover from anesthesia before being returned to housing.

Seven days after ischemic injury animals were sacrificed by CO₂ inhalation and globes dissected with the proximal optic nerves intact. The anterior chamber was removed, and the eyes were fixed for 30 minutes in 4% paraformaldehyde/PBS, followed by overnight incubation in 30% sucrose. Fixed eyes were trimmed of additional connective tissue and cut along a sagittal plane to generate a flat face for embedding, and the exposed retinal edge was attached to the sclera with tissue adhesive. The eye was infiltrated with increasing strengths of JB-4 embedding medium (glycol methacrylate; GMA), followed by embedding in JB-4 at 4° C. for sagittal sectioning. Optic nerves were co-embedded with their paired retina for transverse sectioning. Embedded tissues were sectioned through or near the optic nerve head at a thickness of 2 μm, and collected on slides for further analysis.

For histological analysis of retinas, sections were stained with 1% toluidine blue in absolute ethanol for 40 seconds, and cover slipped for microscopic examination. Images were captured using cellSens Dimension software on an Olympus BX61 upright microscope with a motorized stage, using a 20× objective and a resolution of 169 nm/pixel, and stitched together using the multiple image alignment procedure of the software. Five locations were chosen at random from across the retina, avoiding areas of extreme curvature or extreme histological disruption, and the thickness of the retinal layers was assessed, as follows: ganglion cells (GCL; count of large, round nuclei in layer near retinal surface in 100 μm region centered on chosen location), inner plexiform layer (IPL; distance from nuclei in ganglion cell layer to nuclei in inner nuclear layer), inner nuclear layer (INL), outer plexiform layer (OPL; distance from nuclei in INL to nuclei in outer nuclear layer), outer nuclear layer (ONL), and total retinal thickness (TR distance from retinal surface above nerve fiber layer to edge of ONL). Photoreceptor outer segments beyond the ONL were not included in measurement because of possible damage from postmortem artifactual retinal detachment. Distances and counts in ischemic eyes were normalized to the mean of the naïve contralateral eye and compared by Student's t-test.

Optic nerves were stained with toluidine blue as above, and imaged with a 60× oil objective at a resolution of 56 nm/pixel and stitched together using the multiple image alignment capabilities of the software. Five regions (100 μm2 each) were selected from unoriented optic nerves—one in each of four arbitrary quadrants plus one centrally located region—and optic nerves stained pink by toluidine blue were counted. To account for possible tilt during embedding, optic nerve sections were measured across perpendicular axes (longest and shortest), and density scaled to assume circularity along the short axis. Total optic nerve counts were estimated by using the scaled density and circular area predicted by the short axis, which is numerically equivalent to using unscaled density and area of an oval defined by the two axes.

### Results

The results of the Example are presented in FIGS. 2 to 4 as further described below.

In FIG. 2, the thickness of the retinal layers from eyes subjected to a period of high IOP (an ischemic insult to the retina) were assessed by histological analysis. The thickness of the same retinal layers were compared between eyes subjected to high IOP and the same retinal layer from the healthy contralateral eye of the same animal, which did not receive an ischemic insult with high IOP. The degree of retinal thinning is more pronounced in the inner retina (including the GCL where retinal ganglion cells are found, and the IPL) in eyes subjected to high IOP, and this thinning its blocked by treatment with either brimonidine or Compound A.

Similarly in FIG. 3, the percentage of protection that is provided by Compound A is greater relative to the vehicle, and slightly greater than the effects of brimonidine. In FIG. 4, 100% of the RGC were protected by Compound A subjected to ischemic insult as compared to around 80% of the RGC protected by brimonidine.

These results suggest that Compound A is protective to retinal ganglion cells.

### Example 2

### Compound Synthesis

### 2′,3′-Isopropylidene-N⁶-cyclohexyladenosine

A solution of 6-chloroadenosine (2.58 g) and cyclohexylamine (5 g) in ethanol (20 ml) was heated at reflux for 6 hours then cooled to room temperature. The reaction mixture was concentrated in vacuo and the resultant residue was diluted with water (50 ml) and ethyl acetate (300 ml). The organic layer was separated and the aqueous layer was extracted with ethyl acetate (2×50 ml). The combined organic layers were washed with water (1×30 ml), dried over sodium sulfate, concentrated in vacuo and dried under vacuum to provide N⁶-cyclohexyladenosine as a white solid (2.600 g). N⁶-Cyclohexyladenosine (2.6 g) was diluted with acetone (30 ml) and to the resultant solution was added 2,2-dimethoxypropane (12 ml), followed by D-camphorsulphonic acid (3.01 g) and the mixture was allowed to stir at room temperature for 18 hours. The reaction mixture was concentrated in vacuo and the resultant residue was diluted with ethyl acetate (150 ml), then neutralized to pH 8.0 using saturated aqueous NaHCO₃. The organic layer was separated, dried over sodium sulfate, concentrated in vacuo. The residue was purified twice on the silica gel column using MeOH—CH₂Cl₂ (4:96) as an eluent to provide 2′,3′-isopropylidene-N⁶-cyclohexyladenosine (3.16 g). ¹H NMR (CDCl₃): δ 1.23-1.47 (m, 9H), 1.38 (s, 3H), 1.64 (s, 3H), 1.79-1.81 (m, 1H), 2.04-2.06 (m, 1H), 3.80 (d, J=12 Hz, 1H), 3.96 (d, J=12 Hz, 1H), 4.53 (s, 1H), 5.09-5.16 (m, 2H), 5.80-5.92 (m, 2H), 7.79 (s, 1H), 8.24 (s, 1H), 8.22-8.38 (m, 1H).

### N⁶-Cyclohexyladenosine-5′-O-nitrate (Compound E)

Acetic anhydride (6 ml) was slowly added to a stirred solution of nitric acid (2 g, 63%) at −25° C. (CCl₄—CO₂ bath used for cooling) and the reaction temperature maintained at −7.5 to 0° C. for additional 1 hr. A solution of 2′,3′-isopropylidene-N⁶-cyclohexyladenosine (1.0 g) in acetic anhydride (3 mL) was added slowly. The resultant reaction was allowed to stir at 0 to −5° C. for 2 hour and the mixture was slowly poured slowly into an ice-cold solution of aqueous NaHCO₃ (40 mL) and ethyl acetate (150 mL) and it was allowed to stir for 5 minutes. The organic layer was separated and washed with water, dried over sodium sulfate, and concentrated in vacuo. The residue was diluted with a mixture of TFA (16 mL) and water (4 mL) and the mixture was allowed to stir for 30 minutes at room temperature. The mixture was concentrated in vacuo and the resultant residue was diluted with water (10 mL) and concentrated in vacuo. The residue obtained was diluted with ethyl acetate and washed with saturated aqueous sodium bicarbonate, and the organic layer was dried over sodium sulfate and concentrated in vacuo. The residue was purified on the silica gel column using ethyl acetate hexane (from 40:60 to 20:80 gradient) to provide N⁶-cyclohexyladenosine-5′-O-nitrate (0.150 gm). ¹H NMR (DMSO-D₆): δ 1.08-1.13 (m, 1H), 1.27-1.41 (m, 4H), 1.57-1.83 (m. 6H), 4.12-4.17 (m, 2H), 4.30-4.33 (m, 1H), 5.48 (d, J=5.4 Hz, 1H), 5.60 (d, J=5.7 Hz, 1H), 5.90 (d, J=4.8 Hz, 1H), 7.59 (d, J=8.1 Hz, 1H), 8.16 (s, 1H), 8.29 (s, 1H).

### N⁶-(exo-2-Norbornyl)adenosine-5′-O-nitrate (Compound F)

2′,3′-Isopropylidene-N⁶-exo-norbornyladenosine was prepared following the procedure of 2′,3′-isopropylidene-N⁶-cyclohexyladenosine and used for the subsequent reaction. Acetic anhydride (6 ml) was slowly added to a stirred solution of nitric acid (2 g, 63%) at −25° C. (CCl₄—CO₂ bath used for cooling) and the reaction temperature maintained at −7.5 to 0° C. for additional 1 hr. A solution of 2′,3′-isopropylidene-N⁶-exo-norbornyladenosine (1.2 g) in acetic anhydride (3 mL) was added slowly. The mixture was allowed to stir at 0 to −5° C. for 40 minutes and the mixture was slowly poured slowly into an ice-cold solution of aqueous NaHCO₃ (40 mL). The solution was extracted in dichloromethane. The organic layer was separated and washed with brine, dried over sodium sulfate, and concentrated under vacuo. The residue was purified on the silica gel column using ethyl acetate-hexane (1:1) to provide the desired product (0.245 g) and the starting compound (1.0 g). The nitro product (0.245 g) was diluted in a mixture of TFA (15 mL) and water (5 mL) and the mixture was allowed to stir for 30 minutes at room temperature. It was concentrated under vacuo and diluted with water (10 mL) and concentrated in vacuo. The resultant residue was diluted with ethyl acetate and washed with saturated aqueous sodium bicarbonate. The organic layer was dried over sodium sulfate and concentrated in vacuo. The residue was recrystallized from the mixture of ethyl acetate and hexane to provide N⁶-exo-2-norbornyladenosine-5′-O-nitrate (0.123 gm). ¹H NMR (DMSO-D₆): δ 1.03-1.21 (m, 3H), 1.40-1.56 (m, 3H), 1.58-1.64 (m. 4H), 3.94 (bs, 1H), 4.13-4.17 (m, 1H), 4.30 (bs, 1H), 4.66-4.87 (m, 3H), 5.49 (d, J=5.4 Hz, 1H), 5.62 (d, J=5.4 Hz, 1H), 5.91 (d, J=4.8 Hz, 1H), 7.60 (d, J=6.6 Hz, 1H), 8.20 (s, 1H), 8.31 (s, 1H).

### 2-Chloro-N⁶-cyclohexyladenosine

A mixture of 2,6-dichloroadenosine (1.0 g) and cyclohexylamine (0.926 g) in ethanol (30 ml) was heated at reflux for 6 hours then cooled to room temperature. The mixture was concentrated under vacuo. The residue was purified on the silica gel column using MeOH—CH₂Cl₂ (1:6 to 1:5). The combined fractions were concentrated and dried under vacuum to provide 2-chloro-N⁶-cyclohexyladenosine as a white solid (2.600 g). ¹H NMR (DMSO-D₆): δ 1.12-1.21 (m, 2H), 1.33-1.43 (m, 3H), 1.63-1.86 (m, 6H), 3.57-3.62 (m, 1H), 3.66-3.69 (m, 1H), 3.97 (d, J=3 Hz, 1H), 4.16 (d, J=3.3 Hz, 1H), 4.54 (d, J=5.4 Hz, 1H), 5.08-5.11 (m, 1H), 5.24 (d, J=4.8 Hz, 1H), 5.51 (d, J=5.7 Hz, 1H), 5.85 (d, J=5.7 Hz, 1H), 8.26 (d, J=8.4 Hz, 1H), 8.41 (s, 1H).

### 2-Chloro-2′,3′-isopropylidene-N⁶-cyclohexyladenosine

2-Chloro-N⁶-cyclohexyladenosine (0.5 g) was diluted with acetone (30 ml) and to the mixture was added 2,2-dimethoxypropane (2.04 g), followed by D-camphorsulphonic acid (CSA, 0.272 g). The resultant reaction mixture was allowed to stir at room temperature for 2 hours. Additional CSA (0.2 g) was added and stirred for 2 hours. The mixture was concentrated in vacuo and the resultant residue was diluted with ethyl acetate, then neutralized to pH 8.0 using concentrated aqueous NaHCO₃. The organic layer was separated, dried over sodium sulfate, concentrated under vacuum to provide 2-chloro-2′,3′-isopropylidene-N⁶-cyclohexyladenosine (0.378 g). ¹H NMR (CDCl₃): δ 1.23-1.30 (m, 3H), 1.36-1.44 (m, 1H), 1.63 (s, 3H), 1.68-1.79 (m, 5H), 2.04-2.08 (m, 2H), 3.81 (d, J=5 Hz, 1H), 3.99 (d, J=12.9 Hz, 1H), 4.51 (s, 1H), 5.11 (d, J=5.7 Hz, 1H), 5.15-5.18 (m, 1H), 5.75 (bs, 1H), 5.78 (d, J=4.5 Hz, 1H), 5.96 (bs, 1H), 7.76 (s, 1H).

### 2-Chloro-N⁶-cyclohexyladenosine-5′-O-sulfate sodium salt (Compound G)

2-Chloro-2′,3′-isopropylidene-N⁶-cyclohexyladenosine (0.540 g) was dissolved in DMF (6 ml) and added slowly in to the solution of sulfur trioxide (0.302 g) in DMF (3 ml). The mixture was stirred overnight at room temperature. It was concentrated on ratavaporator and the residue was diluted with water (8 ml). The water solution was slowly neutralized with NaOH (0.1N) to pH 7.0. It was extracted in ethyl acetate and the aqueous layer was then concentrated. The white solid obtained was used as such for the next step. The protected sodium sulfate salt was treated with the mixture of TFA-water (16:4 ml) and stirred for 30 min. The reaction mixture was concentrated and the residue was crystallized from acetone to provide 2-chloro-N⁶-cyclohexyladenosine-5′-O-sulfate sodium salt (0.150 g). ¹H NMR (DMSO-D₆): δ 1.10-1.13 (m, 1H), 1.25-1.41 (m, 4H), 1.57-1.83 (m. 6H), 3.72-4.08 (m, 4H), 4.47 (s, 1H), 5.81 (s, 1H), 8.14 (d, J=6.0 Hz, 1H), 8.43 (s, 1H).

### 2-Chloro-N⁶-cyclohexyladenosine-5′-O-nitrate (Compound H)

Following the nitration and the TFA water deprotection reactions, 2-chloro-N⁶-cyclohexyladenosine-5′-O-nitrate was prepared from 2-chloro-2′,3′-isopropylidene-N⁶-cyclohexyladenosine. ¹H NMR (CDCl₃): δ 1.06-1.42 (m, 4H), 1.64-1.88 (m, 5H), 4.08 (bs, 1H), 4.21 (s, 1H), 4.30 (d, J=4.2 Hz, 1H), 4.41 (s, 1H), 4.83-4.88 (m, 2H), 5.57 (d, J=5.4 Hz, 1H), 5.70 (d, J=4.5 Hz, 1H), 5.90 (d, J=5.1 Hz, 1H), 8.26 (d, J=8.7 Hz, 1H), 8.38 (s, 1H).

### Synthesis of Compound A

### N⁶-Cyclopentyladenosine (Compound I)

A solution of 6-chloroadenosine (43 g) and cyclopentylamine (5 eq.) in ethanol (50 eq.) was heated at reflux for 3 hours then cooled to room temperature. The resultant reaction mixture was concentrated in vacuo and the resultant residue was diluted with water (400 ml) and ethyl acetate (400 ml). The organic layer was separated and the aqueous layer was extracted into ethyl acetate (2×400 ml). The combined organic layers were washed with water (2×200 ml), dried over sodium sulfate, concentrated in vacuo and dried under vacuum to provide a solid which was suspended in MeOH (400 mL), filtered and dried to provide N⁶-cyclopentyladenosine (43.8 g).

### 2′,3′-isopropylidene-N⁶-cyclopentyladenosine

N⁶-cyclopentyladenosine (43 g) was diluted with acetone (75 eq.) and to the resultant solution was added 2,2-dimethoxypropane (5 eq.), followed by D-camphorsulphonic acid (1 eq) and the resultant reaction was allowed to stir at room temperature for 3 hours. The resultant reaction mixture was concentrated in vacuo and the resultant residue was diluted with ethyl acetate, then neutralized to pH 7.0 using concentrated aqueous NaHCO₃. The organic layer was separated, dried over sodium sulfate, concentrated in vacuo and dried under vacuum to provide a solid which was suspended in hexane (250 mL), filtered, washed with hexane and dried under vacuum to provide 2′,3′-isopropylidene-N⁶-cyclopentyl adenosine (43 g).

### 2′,3′-isopropylidene-N⁶-cyclopentyladenosine-5′-nitrate

Acetic anhydride (22 eq) was slowly added to a stirred solution of nitric acid (5 eq., 63%) at −10° C. (acetonitrile-CO₂ bath used for cooling) over a period of 4 hours with the reaction temperature maintained at −5 to 5° C. during the addition. The resultant solution was cooled to −20° C. and a solution of 2′,3′-isopropylidene-N⁶-cyclopentyladenosine (18.250 gm, 0.048 mol) in acetic anhydride (37 mL, 8 eq.) was added slowly. The resultant reaction was allowed to stir at −15 to −5° C. for 1 hour and the resultant reaction mixture was slowly poured slowly into an ice-cold solution of aqueous NaHCO₃ (168 gm in 800 mL water) and ethyl acetate (350 mL) and the resultant solution was allowed to stir for 5 minutes. The organic layer was separated and the aqueous layer was extracted using ethyl acetate (350 mL). The combined organic layers were washed with water, and dried over sodium sulfate, concentrated in vacuo and purified using flash column chromatograpy on silica gel using 70% ethyl acetate-hexane as eluent to provide 2′,3′-isopropylidene-N⁶-cyclopentyladenosine-5′-nitrate (14.9 g).

### Compound A

2′,3′-isopropylidene-N⁶-cyclopentyladenosine-5′-nitrate (4.8 g) was diluted with a mixture of TFA (20 mL) and water (5 mL) and the resultant reaction was allowed to stir for 30 minutes at room temperature. The resultant reaction mixture was concentrated in vacuo and the resultant residue was diluted with water (10 mL) and concentrated in vacuo. The resultant residue was diluted with ethyl acetate and washed with saturated aqueous sodium bicarbonate, and the organic layer was dried over sodium sulfate and concentrated in vacuo to provide a white solid residue which was dried under vacuum and then recrystallized from cold ethanol to provide Compound A (3.1 gm). ¹H-NMR (DMSO-d₆): δ 1.49-1.58 (m, 4H), 1.66-1.72 (m, 2H), 1.89-1.94 (m, 2H), 4.12-4.17 (m, 1H), 4.28-4.33 (m, 1H), 4.48 (bs, 1H), 4.65-4.87 (m, 3H), 5.5 (d, J=5.1 Hz, 1H), 5.63 (d, J=5.7 Hz, 1H), 5.91 (d, J=5.1 Hz, 1H), 7.75 (d, J=7.5 Hz, 1H), 8.17 (bs, 1H), 8.30 (s, 1H); MS (ES⁺): m/z 381.35 (M+1); Anal. Calcd for C₁₅H₂₀N₆O₆: C, 47.37; H, 5.30; N, 22.10. Found: C, 47.49; H, 5.12, N, 21.96.

### Synthesis of Compound B

### 2-Chloro-N⁶-cyclopentyladenosine

2′,3′,5′-triacetoxy-2,6-dichloroadenosine (1.5 g) and cyclopentylamine (8 eq.) were diluted with ethanol (50 eq.) and the resulting solution was heated at reflux for about 15 hours, then cooled to room temperature and concentrated in vacuo to provide a crude residue which was diluted with a mixture of ethyl acetate and water and transferred to a separatory funnel. The organic layer was separated, dried over sodium sulfate and concentrated in vacuo to provide a crude residue which was purified using flash column chromatography on silica gel (8% MeOH-dichloromethane as eluent) to provide 2-chloro-N⁶-cyclopentyladenosine (0.948 g). MS m/z 370.32 [M+H]⁺.

### 2′,3′-Isopropylidene-2-chloro-N⁶-cyclopentyladenosine

2-chloro-N⁶-cyclopentyladenosine (900 mg, as prepared in the previous step) and 2,2-dimethoxypropane (10 eq.) were diluted with acetone (15 mL) and to the resulting solution was added D-camphorsulphonic acid (1 eq.) and the resulting reaction was allowed to stir at room temperature for 2 hr. The resulting reaction mixture was concentrated in vacuo, diluted with a mixture of saturated aqueous NaHCO₃ and ethyl acetate, and transferred to a separatory funnel. The organic layer was separated, dried over sodium sulfate and concentrated in vacuo to provide a crude residue which was purified using flash column chromatography on silica gel (using 5% MeOH-dichloromethane as eluent) to provide 2′,3′-Isopropylidene-2-chloro-N⁶-cyclopentyladenosine (0.905 g). ¹H NMR (CDCl₃, 300 MHz): δ 1.36 (s, 3H), 1.62 (s, 3H), 1.66-2.16 (m, 9H), 3.78 (d, J=12.9 Hz, 1H), 3.98 (d, J=12.9 Hz, 1H), 4.51 (bs, 1H), 4.55-4.60 (m, 1H), 5.09-5.17 (m, 2H), 5.81 (bs, 1H), 7.25 (s, 1H), 7.89 (s, 1H).

### 2′,3′-Isopropylidene-2-chloro-N⁶-cyclopentyladenosine-5′-nitrate

A solution of nitric acid (2.0 mL, 60%) was added slowly over a period of 30 minutes to acetic anhydride (16.0 mL) at −10 to 10° C. (using acetonitrile-CO₂ cooling bath) and the reaction mixture was allowed to stir at −10 to 10° C. for 10 minutes. The reaction mixture was then cooled to −30° C. and then a solution of 2′,3′-Isopropylidene-2-chloro-N⁶-cyclopentyladenosine (655 mg, 0.0016 mol, as prepared in the previous step) in acetic anhydride (8.0 mL) was added slowly. When addition was complete, the resulting reaction was allowed to warm to −5° C. and monitored using TLC (solvent 5% MeOH—CH₂Cl₂ or 70% EtOAc-hexane). When the reaction was complete, the reaction mixture was poured slowly into an ice cold mixture of saturated aqueous NaHCO₃ (300 equivalent in 75 mL water) and ethyl acetate (60 mL). The organic layer was separated and the aqueous layer was back extracted with ethyl acetate. The combined organic layers were washed with water, dried over sodium sulfate, and concentrated in vacuo to provide a crude residue. The crude residue was purified using flash column (5% methanol-dichloromethane as eluent) to provide 2′,3′-Isopropylidene-2-chloro-N⁶-cyclopentyladenosine-5′-nitrate (0.435 g). ¹H NMR (CDCl₃, 300 MHz): δ 1.38 (s, 3H), 1.59 (s, 3H), 1.66-2.13 (m, 9H), 4.50-4.55 (m, 1H), 4.71-4.83 (m, 2H), 5.14-5.17 (m, 1H), 5.31 (d, J=5.7 Hz, 1H), 6.04 (s, 1H), 7.24 (s, 1H), 7.81 (s, 1H). MS m/z 455.44 [M+H]⁺.

### Compound B

2′,3′-Isopropylidene-2-chloro-N⁶-cyclopentyladenosine-5′-nitrate (0.435 g, as prepared in the previous step) was diluted with TFA (20 mL) and water (5 mL) and the resulting solution was allowed to stir for 30 minutes. The resulting reaction mixture was concentrated in vacuo and the resulting residue was diluted with water (10 mL) and the resulting solution was concentrated in vacuo. The crude residue obtained was diluted with ethyl acetate, transferred to a separatory funnel, washed with saturated aqueous sodium bicarbonate, dried over sodium sulfate and concentrated in vacuo. The crude residue obtained was purified using flash column chromatography on silica gel (using 10% methanol-dichloromethane as eluent) to provide Compound 16 (0.250 g). ¹H NMR (DMSO-d₆, 300 MHz): δ 1.52-1.95 (m, 9H), 4.13-4.24 (m, 2H), 4.55-4.58 (m, 1H), 4.73-4.85 (m, 2H), 5.50 (bs, 1H), 5.61 (bs, 1H), 5.84 (d, J=5.1 Hz, 1H), 8.33 (bs, 2H), MS m/z 414.85 [M+H]⁺.

### Synthesis of Compound C (Sodium Salt)

A mixture of 2′,3′-isopropylidene-N⁶-cyclopentyladenosine (1 g, 0.0026 mol, prepared as set forth in Example 1) and sulfur trioxide-pyridine complex (0.0039 mol) in DMF (17 mL) was stirred at room temperature for about 18 hours. The DMF was removed in vacuo and the resulting residue was dried in vacuo. The dried residue was diluted with water (25 mL), neutralized to pH 7.0 using NaOH (1N) and concentrated in vacuo to provide a crude residue which was diluted with a solution of TFA (80% solution in water, 50 mL). The resulting solution was allowed to stir at 25° C. for 30 minutes and the reaction mixture was concentrated in vacuo to afford a crude residue which was diluted with water (10 mL) and concentrated in vacuo. The crude compound obtained was recrystallized from acetone-water to provide compound C (sodium salt) (805 mg). ¹HMNR (DMSO-d₆, 300 MHz): 1.53-1.96 (m, 9H), 3.78-4.10 (m, 4H), 4.43-4.54 (m, 2H), 5.90 (d, J=5.1 Hz, 1H), 8.23 (s, 1H), 8.46 (s, 1H). MS m/z 416.20 [M+H]⁺.

### Example 3

### Binding Studies

### Cell Culture and Membrane Preparation

CHO cells stably transfected with human adenosine A₁ receptor are grown and maintained in Dulbecco's Modified Eagles Medium with nutrient mixture F12 (DMEM/F12) without nucleosides, containing 10% fetal calf serum, penicillin (100 U/mL), streptomycin (100 μg/mL), L-glutamine (2 mM) and Geneticin (G-418, 0.2 mg/mL; A_{2B}, 0.5 mg/mL) at 37° C. in 5% CO₂/95% air. Cells are then split 2 or 3 times weekly at a ratio of between 1:5 and 1:20.

Membranes for radioligand binding experiments are prepared from fresh or frozen cells as described in Klotz et al., Naunyn-Schmiedeberg's Arch. Pharmacol., 357:1-9 (1998). The cell suspension is then homogenized in ice-cold hypotonic buffer (5 mM Tris/HCl, 2 mM EDTA, pH 7.4) and the homogenate is spun for 10 minutes (4° C.) at 1,000 g. The membranes are then sedimented from the supernatant for 30 minutes at 100,000 g and resuspended in 50 mM Tris/HCl buffer pH 7.4 (for A₃ adenosine receptors: 50 mM Tris/HCl, 10 mM MgCl₂, 1 mM EDTA, pH 8.25), frozen in liquid nitrogen at a protein concentration of 1-3 mg/mL and stored at −80° C.

### Adenosine Receptor Binding Studies

The affinities of selected Purine Compounds for the adenosine A₁ receptor can be determined by measuring the displacement of specific [³H]2-chloro-N⁶-cyclopentyl adenosine binding in CHO cells stably transfected with human recombinant A₁ adenosine receptor expressed as Ki (nM).

Dissociation constants of unlabeled compounds (Kᵢ-values) are determined in competition experiments in 96-well microplates using the A₁ selective agonist 2-chloro-N⁶-[³H]cyclopentyladenosine ([³H]CCPA, 1 nM) for the characterization of A₁ receptor binding. Nonspecific binding is determined in the presence of 100 μM R-PIA and 1 mM theophylline, respectively. For details see Klotz et al., Naunyn-Schmiedeberg's Arch. Pharmacol., 357:1-9, 1998. Binding data can be calculated by non-linear curve fitting using the program SCTFIT (De Lean et al. Mol. Pharm. 1982, 21:5-16).

### Functional Characterization

The A₁ and A₃ receptor-mediated inhibition of forskolin-stimulated adenylyl cyclase activity was tested in membranes prepared from CHO cells stably transfected with the human A₁ and A₃ adenosine receptors. The A₂ₐ and A_{2b} receptor-mediated stimulation of basal cyclase activity was tested in membranes prepared from CHO cells stably transfected with the human A₂ₐ and A₃ adenosine receptors.

Adenylyl cyclase inhibition via human adenosine A₁ and A₃ receptors

| | A₁ (EC₅₀ nM) | A₃ (EC₅₀ nM) |
|---|---|---|
| Compound A | 17 | >100,000 |
| Compound B | 20 | >100,000 |
| Compound E | 29 | >100,000 |
| Compound G | 19 | >100,000 |

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

What is claimed is:

1. A method of preventing retinal ganglion cell damage in a subject in need thereof, comprising the step of: applying a pharmaceutical composition comprising an effective amount of a compound according to Formula I to an eye of the subject, [chemical structure]
or a pharmaceutically acceptable salt thereof,
wherein
A is —CH₂ONO₂, —CH₂OH, —CH₂OSO₃H;
B and C are —OH;
D is [chemical structure]
A and B are trans with respect to each other;
B and C are cis with respect to each other;
C and D are cis or trans with respect to each other;
R¹ is —H, —C₁-C₁₀ alkyl, -aryl, -3- to 7-membered monocyclic heterocycle, -8- to 12-membered bicyclic heterocycle, —C₃-C₈ monocyclic cycloalkyl, —C₃-C₈ monocyclic cycloalkenyl, —C₈-C₁₂ bicyclic cycloalkyl, —C₈-C₁₂ bicyclic cycloalkenyl-(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), or —(CH₂)ₙ-aryl;
R² is —H, halo, —CN, —NHR⁴, —NHC(O)R⁴, —NHC(O)OR⁴, —NHC(O)NHR⁴, —NHNHC(O)R⁴, —NHNHC(O)OR⁴, —NHNHC(O)NHR⁴, or —NH—N═C(R⁶)R⁷;
R⁴ is —C₁-C₁₅ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —C≡C—(C₁-C₁₀ alkyl) or —C≡C-aryl;
R⁶ is —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ—(C₃-C₈ monocyclic cycloalkenyl), -phenylene-(CH₂)ₙCOOH, or -phenylene-(CH₂)ₙCOO—(C₁-C₁₀ alkyl);
R⁷ is —H, —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkenyl) or —(CH₂)ₙ—(C₈-C₁₂ bicyclic cycloalkyl);
and each n is independently an integer ranging from 1 to 5, and a pharmaceutically acceptable vehicle.

2. The method of claim 1, wherein the compound of Formula I has the formula: [chemical structure]
or a pharmaceutically acceptable salt thereof,
wherein
A is —CH₂ONO₂;
B and C are —OH;
D is [chemical structure]
A and B are trans with respect to each other;
B and C are cis with respect to each other;
C and D are cis or trans with respect to each other;
R¹ is —C₃-C₈ monocyclic cycloalkyl, -3- to 7-membered monocyclic heterocycle or —C₈-C₁₂ bicyclic cycloalkyl; and
R² is —H or -halo.

3. The method of claim 1, wherein the compound of Formula I is selected from: [chemical structure]
((2R,3S,4R,5R)-5-(6-(cyclopentylamino)-9H-purin-9-yl)-3,4-dihydroxytetrahydrofuran-2-yl)methyl nitrate, [chemical structure]
((2R,3S,4R,5R)-5-(2-chloro-6-(cyclopentylamino)-9H-purin-9-yl)-3,4-dihydroxytetrahydrofuran-2-yl)methyl nitrate, [chemical structure]
sodium ((2R,3S,4R,5R)-5-(6-(cyclopentylamino)-9H-purin-9-yl)-3,4-dihydroxytetrahydrofuran-2-yl)methyl sulfate, [chemical structure]
((2R,3 S,4R,5R)-3,4-dihydroxy-5-(6-(tetrahydrofuran-3-ylamino)-9H-purin-9-yl)tetrahydrofuran-2-yl)methyl nitrate, [chemical structure]
((2R,3 S,4R,5R)-5-(6-(cyclohexylamino)-9H-purin-9-yl)-3,4-dihydroxytetrahydrofuran-2-yl)methyl nitrate, [chemical structure]
((2R,3S,4R,5R)-5-(6-(bicycle-[2.2.1]-heptan-2-ylamino)-9H-purin-9-yl)-3,4-dihydroxytetrahydrofuran-2-yl)methyl nitrate, [chemical structure]
sodium ((2R,3S,4R,5R)-5-(2-chloro-6-(cyclohexylamino)-9H-purin-9-yl)-3,4-dihydroxytetrahydrofuran-2-yl)methyl sulfate, [chemical structure]
((2R,3S,4R,5R)-5-(2-chloro-6-(cyclohexylamino)-9H-purin-9-yl)-3,4-dihydroxytetrahydrofuran-2-yl)methyl nitrate, and [chemical structure]
Cyclopentyladenosine (CPA), [chemical structure]
2-chlorocyclopentyladenosine (CCPA), [chemical structure]
Cyclohexyladenosine (CHA),
or pharmaceutically acceptable salts thereof.

4. The method of claim 3, wherein the compound is selected from
((2R,3S,4R,5R)-5-(6-(cyclopentylamino)-9H-purin-9-yl)-3,4-dihydroxytetrahydrofuran-2-yl)methyl nitrate;
((2R,3 S,4R,5R)-5-(2-chloro-6-(cyclopentylamino)-9H-purin-9-yl)-3,4-dihydroxytetrahydrofuran-2-yl)methyl nitrate;
sodium ((2R,3S,4R,5R)-5-(6-(cyclopentylamino)-9H-purin-9-yl)-3,4-dihydroxytetrahydrofuran-2-yl)methyl sulfate;
((2R,3S,4R,5R)-3,4-dihydroxy-5-(6-(tetrahydrofuran-3-ylamino)-9H-purin-9-yl)tetrahydrofuran-2-yl)methyl nitrate; and
cyclopentyladenosine.

5. The method of claim 1, comprising the step of applying a pharmaceutical composition comprising about 0.1% to about 5.0% (w/v) of a compound according to Formula I from 1 to 4 times daily.

6. The method of claim 1, comprising the step of applying a pharmaceutical composition comprising about 1.0% to about 3.0% (w/v) of a compound according to Formula I from 1 to 2 times daily.

7. The method of claim 1, comprising the step of applying a pharmaceutical composition comprising about 500 μg-1500 μg of a compound according to Formula I from 1 to 2 times daily.

8. The method of claim 5, wherein the compound is administered in drops.

9. The method of claim 8, wherein the compound is administered in 1 to 2 drops.

10. A method of reducing retinal ganglion cell damage in a subject in need thereof, comprising the step of: applying a pharmaceutical composition comprising an effective amount of a compound according to Formula I to an affected eye of the subject, [chemical structure]
or a pharmaceutically acceptable salt thereof,
wherein
A is —CH₂ONO₂, —CH₂OH— or —CH₂OSO₃H;
B and C are —OH;
D is [chemical structure]
A and B are trans with respect to each other;
B and C are cis with respect to each other;
C and D are cis or trans with respect to each other;
R¹ is —H, —C₁-C₁₀ alkyl, -aryl, -3- to 7-membered monocyclic heterocycle, -8- to 12-membered bicyclic heterocycle, —C₃-C₈ monocyclic cycloalkyl, —C₃-C₈ monocyclic cycloalkenyl, —C₈-C₁₂ bicyclic cycloalkyl, —C₈-C₁₂ bicyclic cycloalkenyl-(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), or —(CH₂)ₙ-aryl;
R² is —H, halo, —CN, —NHR⁴, —NHC(O)R⁴, —NHC(O)OR⁴, —NHC(O)NHR⁴, —NHNHC(O)R⁴, —NHNHC(O)OR⁴, —NHNHC(O)NHR⁴, or —NH—N═C(R⁶)R⁷;
R⁴ is —C₁-C₁₅ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —C≡C—(C₁-C₁₀ alkyl) or —C≡C-aryl;
R⁶ is —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ—(C₃-C₈ monocyclic cycloalkenyl), -phenylene-(CH₂)ₙCOOH, or -phenylene-(CH₂)ₙCOO—(C₁-C₁₀ alkyl);
R⁷ is —H, —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkenyl) or —(CH₂)ₙ—(C₈-C₁₂ bicyclic cycloalkyl);
and each n is independently an integer ranging from 1 to 5, and a pharmaceutically acceptable vehicle.

11. The method of claim 10, wherein the compound of Formula I has the formula: [chemical structure]
or a pharmaceutically acceptable salt thereof,
wherein
A is —CH₂ONO₂;
B and C are —OH;
D is [chemical structure]
A and B are trans with respect to each other;
B and C are cis with respect to each other;
C and D are cis or trans with respect to each other;
R¹ is —C₃-C₈ monocyclic cycloalkyl, -3- to 7-membered monocyclic heterocycle or —C₈-C₁₂ bicyclic cycloalkyl; and
R² is —H or -halo.

12. The method of claim 10 wherein the compound of Formula I is selected from: [chemical structure]
((2R,3S,4R,5R)-5-(6-(cyclopentylamino)-9H-purin-9-yl)-3,4-dihydroxytetrahydrofuran-2-yl)methyl nitrate, [chemical structure]
((2R,3S,4R,5R)-5-(2-chloro-6-(cyclopentylamino)-9H-purin-9-yl)-3,4-dihydroxytetrahydrofuran-2-yl)methyl nitrate, [chemical structure]
sodium ((2R,3S,4R,5R)-5-(6-(cyclopentylamino)-9H-purin-9-yl)-3,4-dihydroxytetrahydrofuran-2-yl)methyl sulfate, [chemical structure]
((2R,3S,4R,5R)-3,4-dihydroxy-5-(6-(tetrahydrofuran-3-ylamino)-9H-purin-9-yl)tetrahydrofuran-2-yl)methyl nitrate, [chemical structure]
((2R,3S,4R,5R)-5-(6-(cyclohexylamino)-9H-purin-9-yl)-3,4-dihydroxytetrahydrofuran-2-yl)methyl nitrate, [chemical structure]
((2R,3S,4R,5R)-5-(6-(bicycle-[2.2.1]-heptan-2-ylamino)-9H-purin-9-yl)-3,4-dihydroxytetrahydrofuran-2-yl)methyl nitrate, [chemical structure]
sodium ((2R,3S,4R,5R)-5-(2-chloro-6-(cyclohexylamino)-9H-purin-9-yl)-3,4-dihydroxytetrahydrofuran-2-yl)methyl sulfate, [chemical structure]
((2R,3S,4R,5R)-5-(2-chloro-6-(cyclohexylamino)-9H-purin-9-yl)-3,4-dihydroxytetrahydrofuran-2-yl)methyl nitrate, and [chemical structure]
cyclopentyladenosine (CPA), [chemical structure]
2-chlorocyclopentyladenosine (CCPA), [chemical structure]
Cyclohexyladenosine (CHA),
or pharmaceutically acceptable salts thereof.

13. The method as claimed in claim 12, wherein the compound is selected from
((2R,3S,4R,5R)-5-(6-(cyclopentylamino)-9H-purin-9-yl)-3,4-dihydroxytetrahydrofuran-2-yl)methyl nitrate;
((2R,3 S,4R,5R)-5-(2-chloro-6-(cyclopentylamino)-9H-purin-9-yl)-3,4-dihydroxytetrahydrofuran-2-yl)methyl nitrate;
sodium ((2R,3S,4R,5R)-5-(6-(cyclopentylamino)-9H-purin-9-yl)-3,4-dihydroxytetrahydrofuran-2-yl)methyl sulfate; ((2R,3S,4R,5R)-3,4-dihydroxy-5-(6-(tetrahydrofuran-3-ylamino)-9H-purin-9-yl)tetrahydrofuran-2-yl)methyl nitrate; and
cyclopentyladenosine (CPA).

14. The method of claim 10, comprising the step of applying a pharmaceutical composition comprising about 0.1% to about 5.0% (w/v) of a compound according to Formula I from 1 to 4 times daily.

15. The method of claim 10, comprising the step of applying about a pharmaceutical composition comprising about 1.0% to about 3.0% (w/v) of a compound according to Formula I from 1 to 2 times daily.

16. The method of claim 10, comprising the step of applying a pharmaceutical composition comprising about 500 μg-1500 μg of a compound according to Formula I from 1 to 2 times daily.

17. The method of claim 14, wherein the compound is administered in drops.

18. The method of claim 17, wherein the compound is administered in 1 to 2 drops.

19. The method of claim 1, further comprising prior, simultaneous or sequential, application of a second ocular agent.

20. The method of claim 19, wherein the second ocular agent is selected from the group comprising: β-blockers, prostaglandin analogs, carbonic anhydrase inhibitors, rho-kinase inhibitors, adrenergic α₂ agonists, miotics, neuroprotectants, A₃ antagonists, A2_{A} agonists, ion channel modulators and combinations thereof.

21. A method of preventing reducing or treating retinal ganglion cell damage in a subject by administering a pharmaceutical composition comprising effective amount of a selective adenosine A₁ agonist to an eye of the subject.

22. The method of claim 21, wherein the subject has or is at risk of developing ocular compression, ocular ischemia, ocular trauma (e.g., Purtsher's retinopathy), ocular inflammation, ocular infection, elevated intraocular pressure, diabetes, interruption in the blood circulation to the retinal ganglion cells, ocular malignancy, ocular disease or general ocular deterioration, glaucoma (e.g., normal tension glaucoma, pseudo-exfoliative and pigment dispersion glaucoma, and closed angle glaucoma), ocular ischemic syndrome, malignancy, retinal ischemia (e.g., retinal hypoxia ischemia), retinal vein occlusion, retinal artery occlusion, diabetic retinopathy, age-related macular degeneration, visual loss from retinal detachment, conditions resulting in increased permeability of the blood-retinal barrier (BRB) resulting in fluid accumulation and retinal edema, or combinations thereof.

23. The method of claim 21, wherein the retinal ganglion cell damage is not caused solely by elevated intraocular pressure.

24. The method of claim 21, wherein the selective adenosine A1 agonist is a compound of Formula I, [chemical structure]
or a pharmaceutically acceptable salt thereof,
wherein
A is —CH₂ONO₂, —CH₂OH— or —CH₂OSO₃H;
B and C are —OH;
D is [chemical structure]
A and B are trans with respect to each other;
B and C are cis with respect to each other;
C and D are cis or trans with respect to each other;
R¹ is —H, —C₁-C₁₀ alkyl, -aryl, -3- to 7-membered monocyclic heterocycle, -8- to 12-membered bicyclic heterocycle, —C₃-C₈ monocyclic cycloalkyl, —C₃-C₈ monocyclic cycloalkenyl, —C₈-C₁₂ bicyclic cycloalkyl, —C₈-C₁₂ bicyclic cycloalkenyl-(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), or —(CH₂)ₙ-aryl;
R² is —H, halo, —CN, —NHR⁴, —NHC(O)R⁴, —NHC(O)OR⁴, —NHC(O)NHR⁴, —NHNHC(O)R⁴, —NHNHC(O)OR⁴, —NHNHC(O)NHR⁴, or —NH—N═C(R⁶)R⁷;
R⁴ is —C₁-C₁₅ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —C≡C—(C₁-C₁₀ alkyl) or —C≡C-aryl;
R⁶ is —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ—(C₃-C₈ monocyclic cycloalkenyl), -phenylene-(CH₂)ₙCOOH, or -phenylene-(CH₂)ₙCOO—(C₁-C₁₀ alkyl);
R⁷ is —H, —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkenyl) or —(CH₂)ₙ—(C₈-C₁₂ bicyclic cycloalkyl) each n is independently an integer ranging from 1 to 5.

25. The method of claim 21, wherein the selective A1 agonist is a compound of formula [chemical structure]
or a pharmaceutically acceptable salt thereof,
wherein
A is —CH₂ONO₂;
B and C are —OH;
D is [chemical structure]
A and B are trans with respect to each other;
B and C are cis with respect to each other;
C and D are cis or trans with respect to each other;
R¹ is —C₃-C₈ monocyclic cycloalkyl, -3- to 7-membered monocyclic heterocycle or —C₈-C₁₂ bicyclic cycloalkyl; and
R² is —H or -halo.

26. The method of claim 25, wherein the compound of Formula I is selected from: [chemical structure]
((2R,3S,4R,5R)-5-(6-(cyclopentylamino)-9H-purin-9-yl)-3,4-dihydroxytetrahydrofuran-2-yl)methyl nitrate, [chemical structure]
((2R,3S,4R,5R)-5-(2-chloro-6-(cyclopentylamino)-9H-purin-9-yl)-3,4-dihydroxytetrahydrofuran-2-yl)methyl nitrate, [chemical structure]
sodium ((2R,3S,4R,5R)-5-(6-(cyclopentylamino)-9H-purin-9-yl)-3,4-dihydroxytetrahydrofuran-2-yl)methyl sulfate, [chemical structure]
((2R,3 S,4R,5R)-3,4-dihydroxy-5-(6-(tetrahydrofuran-3-ylamino)-9H-purin-9-yl)tetrahydrofuran-2-yl)methyl nitrate, [chemical structure]
((2R,3 S,4R,5R)-5-(6-(cyclohexylamino)-9H-purin-9-yl)-3,4-dihydroxytetrahydrofuran-2-yl)methyl nitrate, [chemical structure]
((2R,3S,4R,5R)-5-(6-(bicycle-[2.2.1]-heptan-2-ylamino)-9H-purin-9-yl)-3,4-dihydroxytetrahydrofuran-2-yl)methyl nitrate, [chemical structure]
sodium ((2R,3S,4R,5R)-5-(2-chloro-6-(cyclohexylamino)-9H-purin-9-yl)-3,4-dihydroxytetrahydrofuran-2-yl)methyl sulfate, [chemical structure]
((2R,3S,4R,5R)-5-(2-chloro-6-(cyclohexylamino)-9H-purin-9-yl)-3,4-dihydroxytetrahydrofuran-2-yl)methyl nitrate, and [chemical structure]
cyclopentyladenosine (CPA), [chemical structure]
2-chlorocyclopentyladenosine (CCPA), [chemical structure]
Cyclohexyladenosine (CHA),
or pharmaceutically acceptable salts thereof.

27. The method of claim 21, wherein the selective adenosine A1 agonist is selected from:
((2R,3S,4R,5R)-5-(6-(cyclopentylamino)-9H-purin-9-yl)-3,4-dihydroxytetrahydrofuran-2-yl)methyl nitrate;
((2R,3 S,4R,5R)-5-(2-chloro-6-(cyclopentylamino)-9H-purin-9-yl)-3,4-dihydroxytetrahydrofuran-2-yl)methyl nitrate;
sodium ((2R,3S,4R,5R)-5-(6-(cyclopentylamino)-9H-purin-9-yl)-3,4-dihydroxytetrahydrofuran-2-yl)methyl sulfate;
((2R,3S,4R,5R)-3,4-dihydroxy-5-(6-(tetrahydrofuran-3-ylamino)-9H-purin-9-yl)tetrahydrofuran-2-yl)methyl nitrate; and
cyclopentyladenosine (CPA).

28. The method of claim 21, wherein the pharmaceutical composition comprises about 0.1% to about 5.0% (w/v) of the selective A₁ agonist.

29. The method of claim 21, wherein the pharmaceutical composition comprises about 1.0% to about 3.0% (w/v) of the selective A₁ agonist.

30. The method of claims 21, wherein the effective amount of the selective adenosine A1 agonist is administered as a single dose.

31. The method of claim 21, wherein the effective amount of the selective adenosine A1 agonist is administered as a twice daily dose.

32. A method of providing ocular neuroprotection in a subject in need thereof, comprising the step of: applying a pharmaceutical composition comprising an effective amount of a compound according to Formula I to an eye of the subject, [chemical structure]
or a pharmaceutically acceptable salt thereof,
wherein
A is —CH₂ONO₂, —CH₂OH, —CH₂OSO₃H;
B and C are —OH;
D is [chemical structure]
A and B are trans with respect to each other;
B and C are cis with respect to each other;
C and D are cis or trans with respect to each other;
R¹ is —H, —C₁-C₁₀ alkyl, -aryl, -3- to 7-membered monocyclic heterocycle, -8- to 12-membered bicyclic heterocycle, —C₃-C₈ monocyclic cycloalkyl, —C₃-C₈ monocyclic cycloalkenyl, —C₈-C₁₂ bicyclic cycloalkyl, —C₈-C₁₂ bicyclic cycloalkenyl-(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), or —(CH₂)ₙ-aryl;
R² is —H, halo, —CN, —NHR⁴, —NHC(O)R⁴, —NHC(O)OR⁴, —NHC(O)NHR⁴, —NHNHC(O)R⁴, —NHNHC(O)OR⁴, —NHNHC(O)NHR⁴, or —NH—N═C(R⁶)R⁷;
R⁴ is —C₁-C₁₅ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —C≡C—(C₁-C₁₀ alkyl) or —C≡C-aryl;
R⁶ is —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ—(C₃-C₈ monocyclic cycloalkenyl), -phenylene-(CH₂)ₙCOOH, or -phenylene-(CH₂)ₙCOO—(C₁-C₁₀ alkyl);
R⁷ is —H, —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkenyl) or —(CH₂)ₙ—(C₈-C₁₂ bicyclic cycloalkyl);
and each n is independently an integer ranging from 1 to 5, and a pharmaceutically acceptable vehicle.

33. The method of claim 32, wherein the compound of Formula I has the formula: [chemical structure]
or a pharmaceutically acceptable salt thereof,
wherein
A is —CH₂ONO₂;
B and C are —OH;
D is [chemical structure]
A and B are trans with respect to each other;
B and C are cis with respect to each other;
C and D are cis or trans with respect to each other;
R¹ is —C₃-C₈ monocyclic cycloalkyl, -3- to 7-membered monocyclic heterocycle or —C₈-C₁₂ bicyclic cycloalkyl; and
R² is —H or -halo.

34. The method of claim 1, wherein the compound of Formula I is selected from: [chemical structure]
((2R,3S,4R,5R)-5-(6-(cyclopentylamino)-9H-purin-9-yl)-3,4-dihydroxytetrahydrofuran-2-yl)methyl nitrate, [chemical structure]
((2R,3S,4R,5R)-5-(2-chloro-6-(cyclopentylamino)-9H-purin-9-yl)-3,4-dihydroxytetrahydrofuran-2-yl)methyl nitrate, [chemical structure]
sodium ((2R,3S,4R,5R)-5-(6-(cyclopentylamino)-9H-purin-9-yl)-3,4-dihydroxytetrahydrofuran-2-yl)methyl sulfate, [chemical structure]
((2R,3S,4R,5R)-3,4-dihydroxy-5-(6-(tetrahydrofuran-3-ylamino)-9H-purin-9-yl)tetrahydrofuran-2-yl)methyl nitrate, [chemical structure]
((2R,3S,4R,5R)-5-(6-(cyclohexylamino)-9H-purin-9-yl)-3,4-dihydroxytetrahydrofuran-2-yl)methyl nitrate, [chemical structure]
((2R,3S,4R,5R)-5-(6-(bicycle-[2.2.1]-heptan-2-ylamino)-9H-purin-9-yl)-3,4-dihydroxytetrahydrofuran-2-yl)methyl nitrate, [chemical structure]
sodium ((2R,3S,4R,5R)-5-(2-chloro-6-(cyclohexylamino)-9H-purin-9-yl)-3,4-dihydroxytetrahydrofuran-2-yl)methyl sulfate, [chemical structure]
((2R,3S,4R,5R)-5-(2-chloro-6-(cyclohexylamino)-9H-purin-9-yl)-3,4-dihydroxytetrahydrofuran-2-yl)methyl nitrate, and [chemical structure]
Cyclopentyladenosine (CPA), [chemical structure]
2-chlorocyclopentyladenosine (CCPA), [chemical structure]
Cyclohexyladenosine (CHA),
or pharmaceutically acceptable salts thereof.

35. The method of claim 34, wherein the compound is selected from
((2R,3S,4R,5R)-5-(6-(cyclopentylamino)-9H-purin-9-yl)-3,4-dihydroxytetrahydrofuran-2-yl)methyl nitrate;
((2R,3 S,4R,5R)-5-(2-chloro-6-(cyclopentylamino)-9H-purin-9-yl)-3,4-dihydroxytetrahydrofuran-2-yl)methyl nitrate;
sodium ((2R,3S,4R,5R)-5-(6-(cyclopentylamino)-9H-purin-9-yl)-3,4-dihydroxytetrahydrofuran-2-yl)methyl sulfate;
((2R,3 S,4R,5R)-3,4-dihydroxy-5-(6-(tetrahydrofuran-3-ylamino)-9H-purin-9-yl)tetrahydrofuran-2-yl)methyl nitrate; and
cyclopentyladenosine.

36. The method of claim 32, comprising the step of applying a pharmaceutical composition comprising about 0.1% to about 5.0% (w/v) of a compound according to Formula I from 1 to 4 times daily.

37. The method of claim 32, comprising the step of applying a pharmaceutical composition comprising about 1.0% to about 3.0% (w/v) of a compound according to Formula I from 1 to 2 times daily.

38. The method of claim 32, comprising the step of applying a pharmaceutical composition comprising about 500 μg-1500 μg of a compound according to Formula I from 1 to 2 times daily.

39. The method of claim 36, wherein the compound is administered in drops.

40. The method of claim 39, wherein the compound is administered in 1 to 2 drops.

41. A method of providing ocular neuroprotection in a subject by administering a pharmaceutical composition comprising an effective amount of a selective adenosine A1 agonist to an eye of the subject.

42. The method of claim 41, wherein the subject has or is at risk of developing ocular compression, ocular ischemia, ocular trauma (e.g., Purtsher's retinopathy), ocular inflammation, ocular infection, elevated intraocular pressure, diabetes, interruption in the blood circulation to the retinal ganglion cells, ocular malignancy, ocular disease or general ocular deterioration, glaucoma (e.g., normal tension glaucoma, pseudo-exfoliative and pigment dispersion glaucoma, and closed angle glaucoma), ocular ischemic syndrome, malignancy, retinal ischemia (e.g., retinal hypoxia ischemia), retinal vein occlusion, retinal artery occlusion, diabetic retinopathy, age-related macular degeneration, visual loss from retinal detachment, conditions resulting in increased permeability of the blood-retinal barrier (BRB) resulting in fluid accumulation and retinal edema, or combinations thereof.

43. The method of claim 41, wherein the neuroprotection is not required solely because of elevated intraocular pressure.
